(19) United States
(12) Patent Application Publication
(10) Pub. No.: US 2012/0271187 A1
(43) Pub. Date: Oct. 25, 2012

(54) METHOD AND DEVICE FOR MONITORING CARBON DIOXIDE

(76) Inventor: FRANKIE MICHELLE MCNEILL, Scotsdale, AZ (US)

(21) Appl. No.: 13/360,390

(22) Filed: Jan. 27, 2012

Related U.S. Application Data
(60) Provisional application No. 61/436,716, filed on Jan. 27, 2011.
(60) Provisional application No. 61/565,950, filed on Dec. 1, 2011.

(51) Int. Cl. A61B 5/097; A61M 15/08; A61M 16/06

(52) U.S. Cl. 600/532

(57) ABSTRACT

Various embodiments provide a medical device for monitoring carbon dioxide in the exhaled breath from a non-intubated patient. Various embodiments provide methods for monitoring expired carbon dioxide, when a patient is under conscious sedation or is in any situation in which knowledge of respiratory status is useful.

### CROSS REFERENCE TO RELATED APPLICATIONS

The present application claims all benefits of and priority to Provisional Patent Application No. 61/436,716, entitled Method and Device for Monitoring carbon Dioxide, filed on Jan. 27, 2011 and incorporates the disclosure of this provisional application by reference in its entirety.

The present application also claims all benefits of and priority to Provisional Patent Application No. 61/565,950 entitled Method and Device for Monitoring carbon Dioxide, filed on Dec. 1, 2011 and incorporates the disclosure of this provisional application w reference in its entirety.

### BACKGROUND

Generally, when a patient is under conscious sedation or is in any situation in which knowledge of respiratory status is useful, it may be desirable to monitor carbon dioxide levels in the exhaled air. The monitoring of carbon dioxide exhaled by a patient during various medical procedures has become the Standard of Care.

For example, on the recommendation of the American Society of Anesthesiologist's (ASA) Committee on Standards and Practice Parameters, an amendment to the ASA Standards of Basic Anesthetic Monitoring was approved in October 2011, making monitoring of exhaled carbon dioxide the Standard of Care during moderate or deep sedation. The ASA Standards state, in part, that during moderate or deep sedation. the adequacy of ventilation shall be evaluated by the continual observation of qualitative clinical signs and monitoring for the presence of exhaled carbon dioxide unless precluded or invalidated by the nature of the patient, procedure, or equipment.

In another example, the Association of Anesthetists of Great Britain and Ireland (AAGBI) released updated recommendations, in May 2011, for the use of capnoaphy outside the operating room. The AAGBI recommendation states, in part, that continuous capnography monitoring should be used for all anesthetized patients, regardless of the airway device used or the location of the patient, for all patients whose trachea is intubated, for all patients undergoing moderate or deep sedation, including during the recovery period, and for all patients undergoing advanced life support.

In still another example, the American Heart Association (AHA) released the updated 2010 Guidelines for Cardiopulmonary Resuscitation and Emergency Cardiovascular Care. The AHA Guidelines stress the critical importance of the continuous waveform Capuogaphy to assess the quality of CPR and detect the return of spontaneous circulation.

In yet another example, the American Association for Respiratory Care (AARC) also issued updated AARC Guidelines, which recommend capnography/capnometry for verification of artificial airway placement in a patient, assessment of pulmonary circulation and respiratory status of the patient, and optimization of mechanical ventilation.

In general, the monitoring of carbon dioxide exhaled by a patient can be accomplished by inserting an oxygen supply nasal prong or cannula into the patient and directing a portion of the air exhaled to a suitable apparatus for measuring the carbon dioxide in the exhaled air sample. For example, a gas analyzer, such as a capnograph, can monitor the concentration or partial pressure of carbon dioxide in the exhaled air sample.

The accuracy of such a non-invasive analysis of exhaled gases depends on the ability of a sampling system to move the exhaled air sample from the patient to the gas analyzer. The waveform of the concentration of the carbon dioxide is critical for accurate analysis. The actual concentration of carbon dioxide in the exhaled air can be affected by the oxygen supply, which reduces the accuracy of the analysis of the sample by the gas analyzer.

### SUMMARY

Generally, embodiments described herein relate to methods, systems, devices, apparatuses and kits that can be used for improved go or fluid analysis and detection. The various methods, systems, delvices, apparatuses and kits may provide improved functionality in some aspects and/or can be used with other technologies to provide added functionality.

In various embodiments, a medical device can be a monitoring device that enhances detection and accuracy of measured carbon dioxide in exhaled breath from a non-intubated patient, who may be at least one of a nose breather or a mouth breather.

Various embodiments provide an adapter for sampling exhaled breath from a patient. The adapter can comprise a flexible portion comprising an exterior surface and an interior surface, and configured to have a diameter of the exterior surface that is less than a diameter of a hole in an oxygen supply mask configured to supply oxygen to a patient. The adapter can comprise a connector coupled to one end of the flexible tube, and configured to connect to a receiving connector on at least one of another piece of tube and a gas analyzer. The adapter can also comprise a fitting or securing device around the exterior surface of the flexible portion, and configured to adjustably fasten the flexible portion through the hole in the mask, a sampling portion comprising a plurality of holes into and around a distal end portion of the flexible portion, and at least one of the plurality of holes configured to be in communication with an interior portion of the tube, and a shaped tip on the distal end of the flexible portion.

In various embodiments, a portion of the adapter can be formable and non-kinking and may be easily inserted into an artificial nasal airway, artificial oral airway, and/or deep within a nasal passage without kinking or obstructing the adapter. In various embodiments, the adapter can comprise an open and/or a closed tip and can comprise a plurality of holes or pores proximate to the tip, which allow the flow of carbon dioxide into the flexible portion to be directed to a gas analyzer.

In various embodiments, the adapter can comprise a connector, which can be compatible with standard gas sampling lines and/or gas analyzers. For example, the connector can be compatible with standard gas analyzers used in general anesthesia and/or can be compatible with gas sampling lines used with portable carbon dioxide detection monitors. In various embodiments, the adapter may be useful in at least one of in an ICU, in operating rooms, in oral surgery, in dentistry, in an emergency medical situation (in a hospital and/or pre-hospital), in veterinary medicine or any other situation where measurement of gases may be useful or necessary. In various embodiments, the adapter can be used on any of a variety of patients, including adults, pediatrics, infants, neonates, and/or animals.

In various embodiments, the adapter may be configured to fit into or to lock firmly into one or more ventilation holes of a face mask used to provide oxygen to a patient, or any type of oxygen delivery mask. This configuration can provide a more accurate and continuous monitoring of exhaled carbon dioxide, even if a patient becomes restless and moves her head. In one einbodiment, the adapter can also be employed without a mask by placing a perforated end of the tip in one of a nasal passage, or an artificial nasopharyngeal airway, or over an oral passage, or an oropharyngeal airway, and simply taping portion of the adapter to the face of a patient. In one embodiment, the adapter can also be employed without a mask by incorporating the adapter with any nasal cannula configured to provide oxygen to a patient.

Various embodiments provide a method of sampling carbon dioxide in a portion of exhaled air from a patient. The method can comprise coupling an adapter to a tube from a gas analyzer and to an inner portion of a mask on a patient; positioning a sampling portion of the adapter into a nasal passage; monitoring carbon dioxide in a portion of exhaled air from the nasal passage; and improving detection of carbon dioxide concentration it the exhaled air from a patient.

Various embodiments provide an adapter configured to receive a portion of exhaled air from a patient. The adapter can comprise a flexible tube comprising an exterior surface and an interior surface and configured to communicate a flow of the portion of exhaled air to a gas analyzer, and a connector coupled to one end of the flexible tube, and confipred to connect to a receiving connector on the gas analyzer. The adapter can also comprise a manifold coupled to a distal end of the flexible portion and configured to communicate a flow of the portion of exhaled air to the flexible portion. The adapter can comprise a first sampling portion comprising a plurality of holes in fluid communication with the flexible portion and coupled to the manifold, and a second sampling portion comprising a plurality of holes in fluid communication with the tube and coupled to the manifold. In some embodiments, the second sampling portion can be configured in a spoon-like shape comprising the plurality of holes along an inner edge of the spoon-like shape. In one embodiment, the first sampling portion can be configured for placement inside a nasal passage, and the second sampling portion may be configured for placement over a mouth.

### BRIEF DESCRIPTION OF THE DRAWINGS

The present disclosure will become more fully understood from the detailed description and the accompanying drawings, wherein:

FIG. 1 is a diagrammatic view illustrating an anesthesia monitoring system comprising a medical device, according to various embodiments;

FIG. 2A is a side view illustrating a non-limiting example of a medical device in a first position, according to various embodiments;

FIG. 2B is a side view illustrating a non-limiting example of a medical device in a second position, according to various embodiments;

FIG. 3 is an exploded view illustrating an anesthesia monitoring system comprising a medical device, according to various embodiments;

FIG. 4 is a perspective view illustrating a medical device coupled to a mask, according to various embodiments;

FIG. 5 is a diagrammatic view illustrating a non-limiting example of a method of use of a medical device, according to various embodiments;

FIG. 6 is a diagrammatic view illustrating a non-limiting example of a method of use of a medical device according to various embodiments;

FIG. 7 is a diagrammatic view illustrating a non-limiting example of a method of use of a medical device, according to various embodiments;

FIG. 8 is a diagrammatic view illustrating a non-limiting example of a method of use of a medical device, according to various embodiments;

FIG. 9 is a side view illustrating a non-limiting example of a medical device, according to various embodiments;

FIG. 10 is a side view illustrating a non-limiting example of a medical device, according to various embodiments;

FIG. 11 is a diagrammatic view illustrating a non-limiting example of a method of use of a medical device, according to various embodiments;

FIG. 12 is a diagrammatic view illustrating a non-limiting example of a method of use of a medical device according to various embodiments;

FIG. 13 is a diagrammatic view illustrating a non-limiting example of a method of use of a medical device, according to various embodiments;

FIG. 14 is a diagrammatic view illustrating a non-limiting example of a method of use of a medical device, according to various embodiments;

FIG. 15 is a diagrammatic view illustrating a non-limiting example of a medical device having a mouthpiece, according to various embodiments;

FIG. 16 is a fragmented view illustrating a non-limiting example of a medical device, according to various embodiments;

FIG. 17 is a diagrammatic view illustratnig a non-limiting example of a medical device, according to various embodiments;

FIG. 18 is a diagrammatic view illustrating a non-limiting example of a medical device, according to various embodiments;

FIG. 19 is a diagrammatic view illustrating a non-limiting example of a medical device, according to various embodiments;

FIG. 20 is a diagrammatic view illustrating a non-limiting example of a medical device, according to various embodiments;

FIG. 21 is a diagrammatic view illustrating a non-limiting example of a medical device, according to various embodiments;

FIG. 22 is a diagrammatic view illustrating a non-limiting example of a medical device, according to various embodiments;

FIG. 23 is a diagrammatic view illustrating a non-limiting example of a medical device, according to various embodiments;

FIG. 24 is a diagrammatic view illustrating a non-limiting example of an airway, according to various embodiments;

FIG. 25 is a diagrammatic view illustrating a non-limiting example of an airway, according to various embodiments;

FIG. 26 is a diagrammatic view illustrating a non-limiting example of an airway, according to various embodiments; and

FIG. 27 is a diagrammatic view illustrating a non-limiting example of a medical device, according to various embodiments.

### DETAILED DESCRIPTION

The following description is merely exemplary in nature and is in no way intended to limit the various embodiments, their application, or uses. As used herein, the phrase “at least one of A, B, and C” should be construed to mean a logical (A or B or C), using a non-exclusive logical “or.” As used herein, the phrase “A, B and/or C” should be construed to mean (A, B, and C) or alternatively (A or B or C), using a non-exclusive logical “or.” It should be understood that steps within a method may be executed in different order without altering the principles of the present disclosure.

The drawings described herein are for illustration purposes only and are not intended to limit the scope of the disclosed embodiments in any way. The drawings described herein are for illustrative purposes only of selected embodiments and not all possible implementations, and are not intended to limit the scope of any of the various embodiments. It is understood that the drawings are not drawn to scale. For purposes of clarity, the same reference numbers will be used in the drawings to identify similar elements.

As used herein, a “nasal passage” can be any of a nostril, a nare, a nasopharynx, a nasal choana, or any other portion of a nasal cavity, or combinations thereof. As used herein, the term artificial nasal airway can refer to an essentially hollow device, which typically can be placed into a nasal passage, such as, for example an artificial nasopharyngeal airway.

As used herein, an “oral passage” can be any of an oropharyngeal airway when an artificial oral airway is in place, an inside of a mouth, across a mouth, any other portion of an oral cavity, or combinations thereof. As used herein, the term artificial oral airway can refer to an essentially hollow device, which typically can be placed into an oral passage, such as, for example, an oropharyngeal airway.

Embodiments herein generally relate to devices and methods useful for measuring gases from, in or near a living organism, such as a body. For example, the devices and methods can be used for monitoring gases such as carbon dioxide. Current carbon dioxide monitoring techniques and devices have a number of limitations. For example, one of the most popular carbon dioxide monitoring approaches in the spontaneously breathing patient utilizes the nasal cannula with oxygen delivery and carbon dioxide detection. These devices have been less accurate in the past due to one or more of a variety factors, including one or more of the following: 1.) The sampling of carbon dioxide is located on the nasal cannula where the oxygen is also delivered. This creates a dilution of the carbon dioxide sample especially when the patient requires higher levels of oxygen. 2.) The nasal cannula only detects nasal carbon dioxide. If patient is a mouth breather, no detection will take place. 3.)The third problem ariseS when the patient's ventilatory status worsens and the patient requires an artificial airway (oral or nasal) device to assist in normal breathing. The nasal cannula will not adapt to detect carbon dioxide at a time it is needed the most when an oral airway is in place. 4.) In cases where a patient requires an oxygen mask due to needing increased oxygen delivery, practitioners will purchase a nasal cannula with oxygen delivery and carbon dioxide detection with no intention of using the oxygen system. The practitioners' chose the nasal cannula oxygen/carbon dioxide type only because of the cannula's carbon dioxide detection capabilities. When this happens the facility has to purchase 2 devices to get oxygen with a mask and FDA approved carbon dioxide detection. This can be very costly to the facility, and patient. Some embodiments provide improvements over existing technologies because the devices described herein (in some embodiments) can be releasably attached (they can be removable) and/or positioned, extended, bended, flexed, moved, etc. to meet the particular needs of a situation and or patient.

The devices and methods described herein can overcome many of the drawbacks and limitations of existing devices and methodologies. Further, the devices and methods can be used with existing methodologies and devices to overcome their limits and drawbacks. Thus, in some instances, existing technology can continue to be used along with he devices and methods described herein.

Therefore, some embodiments relate generally to devices that are referred to herein as “adapter” devices because in some embodiments, the devices can function to work with existing or other technologies. In some cases the devices can he used to adapt existing or new technologies to overcome their drawbacks or limitations. This can provide better analysis, but also can be economically important because it allows use of existing resources and materials.

In some embodiments, the adapter devices can have a unique design allowing for improved exhaled carbon dioxide monitoring and will alleviate one or more of the above problems.

For example, in some embodiments the design of an adapter can allow for enhanced detection of carbon dioxide due to the adapter's flexibility and adjustability with the nasal passage. The adapters can be safely placed anywhere in the nasal passageway from the edge of the nasal passageway to the deep posterior nasal passageway or anywhere in between, for example. This can allow a practitioner to adjust the level within the nasal passageway so that he or she gets the best sampling of carbon dioxide. To those skilled in the art, this is detected by observing the waveforms through capnography technology. In some embodiments the adapter devices are safe enough to be placed deep in the posterior nasal passageway, for example, in an area known as the “choana.” The choana is a funnel shaped area between the two posterior nasal passageways; it is located where the back of nasal passages meet and opens into the nasopharynx. This space can allow for less diluted sanipling of carbon dioxide due to its position closer to the trachea and thus the lungs. At present the nasal cannulas with carbon dioxide detection measure carbon dioxide at the edge of the anterior nasal passageway furthest from the trachea, where the carbon dioxide may be more diluted with oxygen.

Also, in some embodiments the adapters can fit to any standard oxygen mask or nasal cannula that is attached to the patient, which can allow for a more continuous unintempted sampling of carbon dioxide even when a patient becomes restless and moves her head about. Furthermore, in some embodiments, the adapter can provide versatility in monitoring sites outside of the nasal passageway, as well as within the nasal passageway. It can be used over a mouth when a patient is mouth breathing. The dual detection model allows for multiple monitoring sites (if desired), for example, in the nasal passageway, mouth, or in the mask (ambient carbon dioxide).

Additionally, in situations where ventilatory status worsens and an artificial oral or a nasal airway device is needed, the devices (e.g., adapters) can function and work with (e.g., fit into) these artificial oral or nasal airway devices to detect carbon dioxide in this real time of need, for example, during respiratory emergencies and CPR, making the detection of the return of spontaneous respiration easier. In some aspects, the adapter can interface with existing technologies without the need of buying new systems to improve the detection of carbon dioxide. The adapter can be used with standard style masks and nasal cannulas. In some aspects they also can be used by simply attaching to the face, for example by taping to the face. The devices and methods are described in detail herein.

It should be understood and appreciated that although the systems, devices/apparatuses and methods are discussed primarily in the context of carbon dioxide detection and analysis, other gases and fluids also can be analyzed, measured and/or detected, such as, for example, oxygen, nitrous oxide, nitrogen, and other such gases and combinations of gases.

According to various embodiments, an adapter, an apparatus, a device, a system andlor a method, as described herein, connecting a tube from a monitoring apparatus to an oxygen supply mask increases the accuracy of carbon dioxide detection from air exhaled from a patient. According to various embodiments, an adapter, as described herein, connecting a tube from a monitoring apparatus to an oxygen supply mask increases the accuracy of carbon dioxide detection from an oropharyngeal airway, for example, with an artificial oral airway in place. According to various embodiments, an adapter, as described herein, connecting a tube from a monitoring apparatus to a oxygen supply mask increases the accuracy of carbon dioxide detection from a nasopharynx, for example, with the artificial nasal airway in place.

According to various embodiments, an adapter, as described herein, connecting a tube from a monitoring apparatus to an oxygen supply mask increases the accuracy of carbon dioxide detection from a nasopharynx when inserted alone into a deep nasal passage or nasal choana. According to various embodiments, an adapter, as described herein, connecting a tube from a monitoring adapter to an oxygen supply mask increases the accuracy of carbon dioxide detection from ambient oral exhaled carbon dioxide when placed across the mouth/lips. According to various embodiments, an adapter, as described herein, connecting a tube from a monitoring adapter to an oxygen supply mask increases the accuracy of carbon dioxide detection from ambient nasal exhaled carbon dioxide when placed in the nare or near the shallow nare.

According to various embodiments, an adapter, as described herein, connecting a tube from a monitoring apparatus to an oxygen supply mask provides an improved waveform of carbon dioxide concentration in exhaled air from a patient. As known to those skilled in the art, an improved waveform provides a more accurate carbon dioxide concentration reading. In one embodiment, an adapter is deployable for nasopharynx carbon dioxide sampling and/or monitoring. In one embodiment, an adapter is deployable for carbon dioxide sampling and/or monitoring in the nasal choana area of a patient. In one embodiment, an adapter is deployable, when an artificial oral airway is in place, for oropharyngeal carbon dioxide sampling and/or monitoring.

In some embodiments, an adapter, as described herein, can also be deployed without an oxygen supply mask in a nasal passage or an oral opening or both, by placing a perforated end of the adzIpter into one of a nasal passage, an artificial nasopharyngeal airway, a nasal choana, in an area near or in an oral passage, or an artificial oropharyngeal airway and then taping a portion of the adapter to the face of a patient. In one embodiment, an adapter, as described herein, can be deployed, without an oxygen supply mask, in a nasal passage and by placing a perforated end of the adapter into a nasal choana and then taping a portion of the adapter to the face of a patient. In one embodiment, the adapter can be connected to an oxygen supply nasal cannula.

It can be appreciated by those skilled in the art, that during medical procedures involving conscious sedation or in any situation in which knowledge of respiratory status is useful, it is desirable to monitor the exhaled air stream from a patient and deliver a portion of such exhaled air stream to a proper monitoring apparatus such as a gas analyzer, mass spectrometer, or capnograph. In various embodiments, an adapter connecting a tube from a monitoring apparatus to an oxygen supply face mask can optimize the repeated samplings taken of the exhaled air stream from a patient to provide aim accurate measurement of carbon dioxide levels.

In various embodiments, when an adapter is employed for monitoring carbon dioxide in a nasal passage, the adapter can be placed deep in it nasal cavity for improved nasopharynx sampling, in which the exhaled carbon dioxide may be more concentrated than in a shallow nare area, which may be near an oxygen supply region, and therefore more accurate than shallow nasal area sampling. For example, nasopharynx sampling may be typically more accurate than shallow nare area sampling due to a high flow of oxygen in the oxygen supply mask fitted on the patient, which can dilute carbon dioxide levels.

In various embodiments, when an adapter is employed for monitoring a mouth of a patient, the adapter can be placed deep in an oral passageway when an artificial oral airway is in place for oropharyngeal airway sampling in which the exhaled carbon dioxide may be more concentrated than at an ambient mouth area, which may be near an oxygen supply region, and therefore provide more accurate ambient mouth sampling. For example, oropharyngeal airway sampling may be more accurate than ambient mouth sampling due to a high flow of oxygen in the oxygen supply mask fitted on the patient. In various embodiments, the adapter can be employed for both nasopharnyx airway sampling and oropharyngeal airway sampling.

In various embodiments, the adapter does not comprise a fitting. In such embodiments, the adapter can be placed between the mask and a skin surface, which is especially advantageous when the mask does not comprise any ventilation holes. In one embodiment, the adapter can be affixed or attached or coupled to the mask with a fastener, which can be, for example, a clip, a clamp, an adhesive strip, a hook and loop connector, a vise, bracket, clasp, snap, connector, link, tie, or combinations thereof.

In various embodiments, an adapter can comprise one of a single catheter, or a dual tube catheter or a triple tube catheter. In one embodiment, a plurality of catheters allow for one or more areas of detection of carbon dioxide in exhaled breath, in any combination a health care professional deems prudent. In various embodiments, the adapter can monitor carbon dioxide in one or more of a nasal passage, an artificial nasopharyngeal airway, an oral passage, an artificial oropharyngeal airway or air within a mask. The adapter can be deployed for monitoring end-tidal carbon dioxide (ETCO2) in a non-intubated patient.

In some embodiments, an adapter, as described herein, can also be deployed without an oxygen supply mask in a nasal passage and over an oral opening or both, by placing a perforated end of the adapter in one of a nasal passage, or an artificial nasopharyngeal airway, a nasal choana, and an area near or in an oral passage, or an oropharyngeal airway. In some embodiments, a first perforated end of the adapter can be positioned into a nasal passage and a second perforated end of the adapter can be positioned near an oral passage. In one embodiment, the second perforated end is replaced by a mouthpiece. In accordance with this embodiment, the first perforated end is positioned in the nose and the mouthpiece is positioned over and/or near the mouth. In some embodiments, a portion of the adapter is taped to the face of a patient. In some embodiments, the adapter can be connected to an oxygen supply nasal cannula.

Various embodiments provide systems for sainpliug exhaled breath from a patient. The systems can comprise a flexible portion comprising an exterior surface and an interior surface, and configured to have a diameter of the exterior surface that is less than a diameter of a hole in an oxygen supply mask configured to supply oxygen to a patient. The system can comprise a connector coupled to one end of the flexible portion, and configured to connect to a receiving connector on at least one of another piece of tube and a gas analyzer. The system can also comprise a fitting or a multi-piece fitting around the exterior surface of the flexible portion, and configured to adjustably fasten the flexible portion through a hole, a sampling portion comprising a plurality of holes into and around a portion of the flexible portion, and at least one of the plurality of holes configured to be in communication with an interior portion of the flexible portion, and a shaped tip on the distal end of the flexible portion.

In one embodiment, the adapter can comprise soft, hollow, and/or humidity absorbent tubing. In various embodiments, the adapter can comprise an open and/or a closed tip and can comprise a plurality of holes or pores proximate to the tip, which allow the flow of carbon dioxide into tube and directed to a gas analyzer. In one embodiment, the adapter can comprise a sensor configured to detect carbon dioxide.

In some embodiments, the adapter can further comprise at least a portion of formable tubing integrated into at least a portion of the flexible portion between the connector and the sampling portion, and the portion of formable tubing can be configured with shape memory to hold a shape formed in the portion of formable tubing. In one embodiment, the portion of formable tubing can comprise an exterior diameter essentially equal to the exterior diameter of the flexible portion and an interior diameter essentially equal to an interior diameter of the flexible portion. In some embodiments, the sampling portion can comprise an exterior diameter essentially equal to the exterior diameter of the flexible portion and an interior diameter essentially equal to or greater than an interior diameter of the flexible portion.

In some embodiments, the system can thither comprise a dryer in a portion of the interior surface of the flexible portion and the dryer can be configured to remove a portion of moisture in the exhaled breath from the patient. In some embodiments, the shaped tip at the distal end of the flexible portion comprises an essentially smooth exterior surface, and comprises a gradient exterior shape from a high center point to a plurality of lower circumference points. In one embodiment, the shaped tip can comprise a weight, which can be configured to lead the tip through a nasal passage for placement of the sampling portion into the nasal passage. In one embodiment, the shaped tip can comprise one or more holes configured to be in communication with the interior portion of the flexible portion. In some embodiments, the sampling portion can be configured for placement into a portion of a nasal passage. In some embodiments, the flexible portion is configured to commtunicate a portion of the exhaled air to the gas analyzer, which is configured to monitor carbon dioxide concentration. In one embodiment, the connector and the fitting are integrated together.

In various embodiments, the system can comprise a y-shaped tithe connecting the sampling portion to the tube and connecting a second sampling portion to the tube. In some embodiments, the system can further comprise a flexible portion integrated between at least one of the y-shaped tube and the sampling portion and between the y-shaped tube and the second sampling portion, wherein the flexible portion-can be configured with shape memory to hold a shape formed in the flexible portion. In some embodiments, the second sampling portion can be configured in a spoon-like shape comprising a plurality of holes in communication with the y-shaped tube and can be configured with the plurality of holes along an inner edge of the spoon-like shape. In some embodiments, the sampling portion can be configured for placement inside a nasal passage, and the second sampling portion can be configured for placement over a mouth.

Various embodiments provide an adapter configured to receive a portion of exhaled air from a patient. The adapter can comprise a flexible tube comprising an exterior surface and an interior surface and configured to communicate a flow of the portion of exhaled air to a gas analyzer, and a connector coupled to one end of the flexible tube, and configured to connect to a receiving connector on the gas analyzer. The adapter can also comprise a manifold coupled to a distal end of the flexible portion and Configured to communicate a flow of the portion of exhaled air to the flexible portion. The adapter can comprise a first sampling portion comprising a plurality of holes in fluid communication with the flexible portion and coupled to the manifold, and a second sampling portion comprising a plurality of holes in fluid communication with the flexible portion and coupled to the manifold.

In some embodiments, the second sampling portion can comprise the plurality of holes around a hollow cylinder at an end distal to the manifold and having an exterior diameter essentially equal to the exterior diameter of the tube and an interior diameter essentially equal to or greater than an interior diameter of the tube, and can comprise a shaped tip capping the end distal from the manifold and having an essentially smooth exterior surface, and comprises a gradient exterior shape from a high center point to a plurality of lower circumference points. In some embodiments, the first sampling portion is configured for placement into a nasal passage. In some embodiments, the second sampling portion can be configured in a spoon-like shape comprising the plurality of holes along an inner edge of the spoon-like shape. In one embodiment, the first sampling portion can be configured for placement inside a nasal passage, and the second sampling portion is configured for placement over a mouth.

In some embodiments, the adapter can comprise a flexible portion integrated between at least one of the manifold and the first sampling portion and between the manifold and the second sampling portion, wherein the flexible portion is configured with shape memory to hold a shape formed in the flexible portion. In some embodiments, the flexible portion can comprise an exterior diameter essentially equal to the exterior diameter of the tube and an interior diameter essentially equal to an interior diameter of the tube. In some embodiments, at least one of the first sampling portion and the second sampling portion comprises an exterior diameter essentially equal to the exterior diameter of the tube and an interior diameter essentially equal to or greater than an interior diameter of the tube. In some embodiments, the adapter can comprise a fastener, which is configured to affix a portion of the adapter to oxygen supply nasal cannula. In one embodiment, the adapter can further comprise the oxygen supply nasal cannula.

Various embodiments provide a method of sampling carbon dioxide in a portion of exhaled air from a patient. The method can comprise coupling an adapter to a tube from a gas analyzer to an inner portion of a mask on to patient; positioning a sampling portion of the adapter into a nasal passage; monitoring carbon dioxide in a portion of exhaled air from the nasal passage; and improving a waveform shape of carbon dioxide concentration in the exhaled air from a patient.

In some embodiments, the method can further comprise positioning a second sampling portion of the adapter over a mouth area of the patient, and monitoring carbon dioxide in a portion of exhaled air fibril the mouth area. In some embodiments, the method can further comprise bending at least a portion of the adapter into a shape and holding the shape in the adapter. In some embodiments, the method can further comprise coupling a fitting configured into the adapter into a hole in the mask. In some embodiments, the method can further comprise removing a portion of moisture in the exhaled air with a dryer configured into the adapter. In some embodiments, the method can further comprise adjusting a position of the sampling portion of the adapter in the nasal passage. In some embodiments, the method can further comprise optimizing detection of the carbon dioxide concentration in the exhaled air from the patient.

In various embodiments, adapter comprises a unique design for improved gas sampling, for example carbon dioxide, of exhaled breath in a spontaneous breathing patient. In various embodiments, the adapter can be connected to any oxygen mask, thus connected to the patient and allowing for adjustability and flexibility of different sites for monitoring and/or detecting carbon dioxide in exhaled breath from the patient.

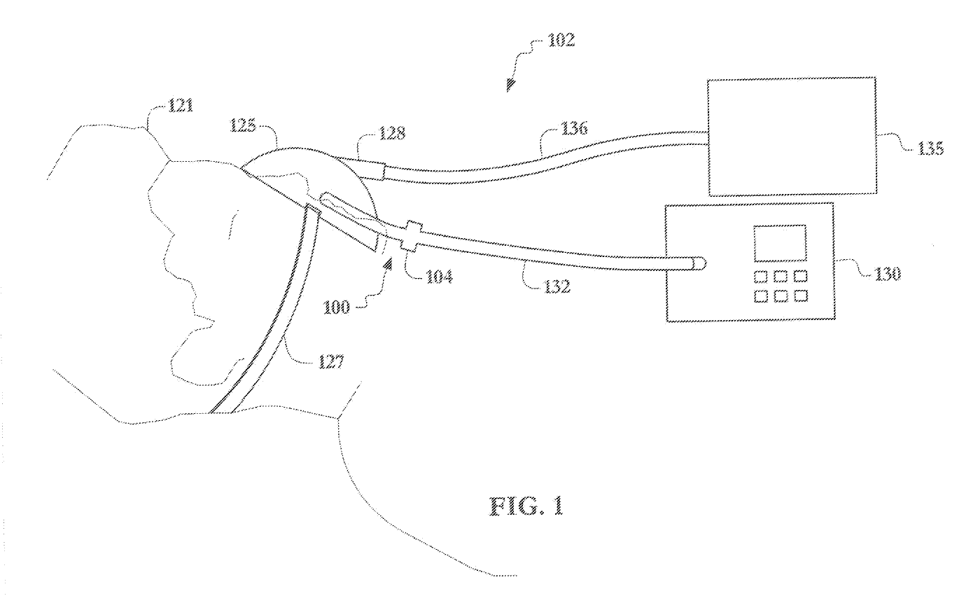

Reference to FIG. 1, anesthesia monitoring system 102 is illustrated, according to various embodiments. Anesthesia monitoring system 102 comprises gas analyzer 130 coupled to oxygen supply mask 125 and oxygen source 135 coupled to mask 125. Mask 125 can be fitted on patient 121 during a medical procedure. As is appreciated by those skilled in the art, oxygen source 135 controls a flow of the oxygen to mask 125 to provide patient 121 with adequate oxygen during a medical procedure or a period of illness. Oxygen source 135 can be coupled to oxygent connector 128 of mask 125 via oxygen line 136.

As will be appreciated by those skilled in the art, gas analyzer 130 can be any of a carbon dioxide monitor, mass spectrometer, or a capnograph. According to various embodiments, gas analyzer 130 monitors at least one of an amount and a concentration of carbon dioxide exhaled by patient 121. In one embodiment, gas analyzer 130 monitors carbon dioxide exhaled by patient 121. Gas analyzer 130 can be configured to analyze carbon dioxide exhaled by patient 121 and any other gas that is either provided to patient 121 or exhaled by patient 121.

According to various embodiments, gas analyzer 130 is coupled to mask 125 via carbon dioxide sample line 132, which is connected to adapter 100 at connector 104 and adapter 100 is interfaced with mask 125. According to various embodiments, a “medical device,” as described herein, can be the adapter, as described herein. In one embodiment, adapter 100 may be referred to as carbon dioxide sampling line adapter. In various embodiments, an “apparatus” or a “device,” as described herein, can be adapter, as described herein.

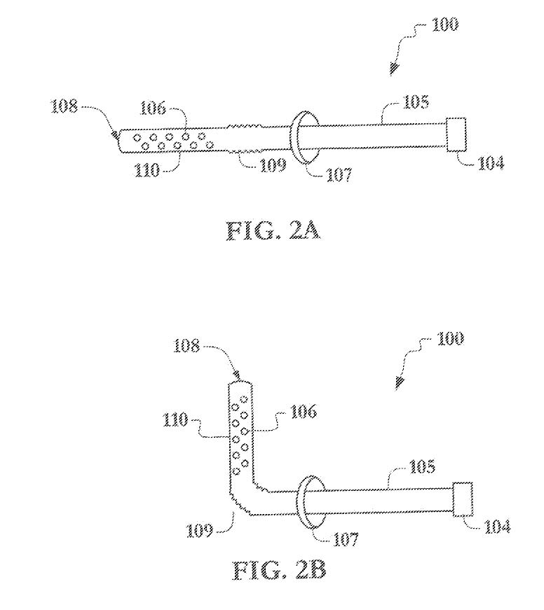

With reference to FIGS. 2A and 2B, adapter 100 is illustrated. According to various embodiments, adapter 100 comprises connector 104 configured to detachably connect to carbon dioxide sample line 132. In one embodiment, connector 104 comprises a male connector configured to couple with a female connector on carbon dioxide sample line 132. In one embodiment connector 104 comprises a female connector configured to couple with a male connector on carbon dioxide line 132. In one embodiment, connector 104 comprises a Luer Lok® Lock connector, which is well blown to those skilled in the art. In various embodiments, connector 104 can be configured to interface or couple to any connector on carbon dioxide sample line 132. In some embodiments, connector 104 can connect directly to gas analyzer 130.

In various embodiments, connector 104 can be configured to hold a line filter (not illustrated). As used by those skilled in the art a line filter may be employed to minimize water vapor from entering gas analyzer 130. In one embodiment, connector 104 is configured in multiple parts, for example, connector 104 may have a threaded coupling around its diameter. Access to the line filter can be accomplished by twisting connector 104 around the threaded coupling to unseat connector 104 into two parts which surround the line filter. In one embodiment, the line filter is in a portion of tubing 105 between connector 104 and fitting 107. In some embodiments, a line filter is replaced with a portion of water absorbing tubing. In some embodiments, the function of a line filter is performed using a Nafion® gas dryer and without a line filter. In one embodiment, at least a portion of adapter 100 comprises Nafion® tubing, which is configured to absorb water in the internal surface of the tubing 105. In one embodiment, tubing 105 is configured to absorb water in the internal surface of the tubing 105.

As illustrated in FIGS. 2A and 2B, connector 104 is coupled to tubing 105. In one embodiment, connector 104 and tubing 105 are separate components with connector 104 configured to he seated around tubing 105. In one embodiment, connector 104 is fused to tubing 105. Also as illustrated in FIGS. 2A and 2B, tithing 105 interfaces with fitting 107. In various embodiments, fitting 107 is configured to interface with mask 125, as described herein. In some embodiments, fitting 107 holds tubing 105 in one of a plurality of ventilation holes (e.g. holes 126 of mask 125 as shown in FIG. 4). In one embodiment, connector 104 can also function as fitting 107. In some embodiments, connector 104 has an outer diameter that is smaller than the diameter of hole 126, which allows connector 104 to be pushed through hole 126 from the inside of mask 125 to mate with carbon dioxide sample line 132. In this embodiment, connector 134 may operate as fitting 107 or as a portion of fitting 107.

Fitting 107 can have any suitable shape or geometry, including but not limited to the geometry as depicted in the various figures. The fitting 107 can be a single member or can be multiple members with any of a number of different geometries. For example, the fitting 107 can function to releasably secure adapter 100 to a mask 125 or into place on the patient 121. It can include one or more bumps or protusions, etc. with valleys or depressions in between that cause the device to remain connected to or in a desired position on the mask 125 or on the patient 121. For example, the bumps can have a diameter that is at least slightly larger than the diameter of the hole 126 in the mask 125 through which it passes so that added force is required for the adapter 100 to pass over and advance beyond the bump or protrusion. As such, the fitting(s) 107 can allow the adapter 100 to be secured into a desired position so that the receiving end of adapter 100 with “perforations” or holes 106 can be in the desired location (e.g., deep in the nasal or oral passageway, outside the mouth or nose, just inside the mouth or nose, etc.).

In some embodiments, at an end of tubing 105 distal to connector 104 is flexible portion 109. In some embodiments, flexible portion 109 is constructed from a material which is flexible and can have enough elasticity to be bent into a position. For example, such a material can be flexible enough to bend but not crimp flexible portion 109 and in some examples may be able to keep the shape of the bend in flexible portion 109 for a period of time. Operating temperature ranges of flexible portion 109 are typically around room temperature, such as, 20° C. to 40° C. however, flexible portion 109 may have operating temperature ranges of 0° C. to 40° C. or 0° C. to 45° C., or −20° C. to 45° C. Although operating temperature ranges of flexible portion 109 are those typically used in operating rooms, flexible portion 109 can be configured to meet extreme operating temperatures, such as those that may be encountered in military hospitals, or in arctic environments, or in outer space, or in a tropical region.

The some embodiments, flexible portion 109 is constructed from a material which is both flexible and has shape memory. In one embodiment, flexible portion 109 comprises enough elasticity to be bent into a position and enough rigidity to maintain the position over a period of time. For example, such a material can be flexible enough to bend but not crimp flexible portion 109 and should be able to keep the shape of the bend in flexible portion 109 for a period of time, for example, at least 5 minutes, or at least 15 minutes, or at least 30 minutes, or at least 45 minutes, or at least an hour, or multiple hours, or 1 day, or 1 month, or multiple months, or at least a year. Operating temperature ranges of flexible portion 109 are typically around room temperature, such as, 20° C. to 40° C., however, flexible portion 109 may have operating temperature ranges of 0° C. to 40° C., 0° C. to 45° C. or −20° C. to 45° C. Although operating temperature ranges of flexible portion 109 are those typically used in operating rooms, flexible portion 109 can be configured to meet extreme operating temperatures, such as those that may be encountered in military hospitals, or in arctic environments, or in outer space, or in a tropical region.

Various embodiments, as illustrated in the Figures, flexible portion 109 is coupled to perforated tube 110. In one embodiment, flexible portion 109 and perforated tube 110 are separate components, which are at least one of mechanically, physically, and chemically attached to one another. In one embodiment, flexible portion 109 and perforated tube 110 are fused together as a continual piece. In various embodiments, adapter 100 can comprise tubing comprising, for example, PTFE, or PEEK, or Tygon, or PVC, or silcone, or KetaSpire, or Radel, or Ixef, or Nafion or any combination thereof. In one embodiment, adapter 100 comprises anti-bacterial tubing. In various embodiments, adapter 100 comprises material that has been approved by the FDA. In some embodiments, adapter 100 comprises material which can withstand sterilization at elevated temperatures. In various embodiments, adapter 100 can comprise tubing which is biocompatible. In various embodiments, adapter 100 can comprise tubing which is typically used in airways. Those skilled in the art will appreciate that choice of materials to construct adapter 100 may be determined based on any one or more of application, price, available materials, and the like.

In one embodiment, at least a portion of flexible portion 109 comprises Nafion® tubing, which is configured to absorb water in the internal surface of the tubing. In one embodiment, flexible portion 109 is configured to absorb water in the internal surface of the flexible portion 109. In one embodiment, flexible portion 109 can be moved in any direction. A standard bendable straw having a flexible portion 109 that can maintain its shape illustrates an example of one embodiment of the mechanics of operation of flexible portion 109. In one embodiment, at least a portion of flexible portion 109 can be corrugated. In one embodiment, flexible portion 109 can be concertina-type hinge between perforated tube 110 and tubing 105. As will be apparent to those skilled in the art, flexible portion 109 and tubing 105 can comprise the same material and may be indistinguishable from each other, except for the location of each of flexible portion 109 and tube 105 in adapter 100.

Perforated tube 110 comprises a plurality of holes 106 which are in communication through adapter 100 to gas analyzer 130. The term “perforated” is used herein, but should not be considered limiting, but refers to any suitable “opening” or series of openings on adapter 100 for receiving a gas that is to be analyzed. For example, the perforations can be one or more holes, slits, apertures, openings, membranes, etc. of any shape, size or number. In one embodiment, plurality of holes 106 can be a plurality of pores in a membrane which is coupled to a portion of perforated tube 110. In one embodiment, perforated tube 110 comprises tip 108, which can be hollow. In one embodiment, tip 108 is in communication through adapter 100 to gas analyzer 130. Various embodiments, at least one of plurality of holes 106 and tip 108 is configured to receive a gas exhaled by the patient 121, which can be sampled by gas analyzer 130. In one embodiment, at least one of plurality of holes 106 and tip 108 is configured to transfer carbon dioxide exhaled by patient 121 to gas analyzer 130. In one embodiment, tip 108 is closed or is solid. In one embodiment, tip 108 can comprise at least one hole 106 in a portion of tip 108 which is protected from nasal material entering holes as adapter 2100 is being pushed into nasal passage 165. For example, tip 108 may comprise a plurality of holes 106 in a surface closest to the perforated tube 110. Tn another example, tip 108 can be mushroom-shaped having a circumference at tip 108 which is larger that a circumference of perforated tube 110. In this example, a plurality of holes 106 can be positioned in tip 108 and be configured to communicate with gas analyzer 130. In this example, a plurality of holes 106 can be positioned in a surface of tip 108 which is closest to perforated tip 108, and be configured to communicate with gas analyzer 130.

In various embodiments, tip 108 comprises at least one of soft edges, rounded edges, and chamfered edges, which can minimize damage to tissue as adapter 100 is placed in an airway. In one embodiment, tip 108 is shaped having soft edges. In one embodiment, tip 108 is shaped having rounded edges. In one embodiment, tip 108 is shaped haying chamfered edges. In one embodiment, at least one tip 108 and perforated tribe 110 comprises a balloon. In one embodiment, at least one of tip 108 and perforated tube 110 is weighted, which can assist in at least one of placing the adapter 100 in an airway and keeping adapter 100 positioned in a nasal passage or an artificial oral airway while patient 121 is breathing. In some embodiments, tip 108 can be shaped having an essentially smooth exterior surface, and comprises a gradient exterior shape from a high center point to a plurality of lower circumference points. In one embodiment, tip 108 can comprise a weight, which can be configured to lead tip 108 through a nasal passage for placement of perforated tube 110 into the nasal passage. In one embodiment, the shaped tip 108 can comprise one or more holes 106 configured to be in communication with the interior portion of the perforated tube 110. In one embodiment, adapter 100 can comprise a balloon coupled to a portion of adapter 100 and configured to secure portion of adapter 100 in a nasal passage of patient 121. In one embodiment, adapter 100 can comprise a weighted member coupled to a portion of adapter 100 and configured to secure a position of adapter 100 in a nasal passage or an artificial oral airway of patient 121.

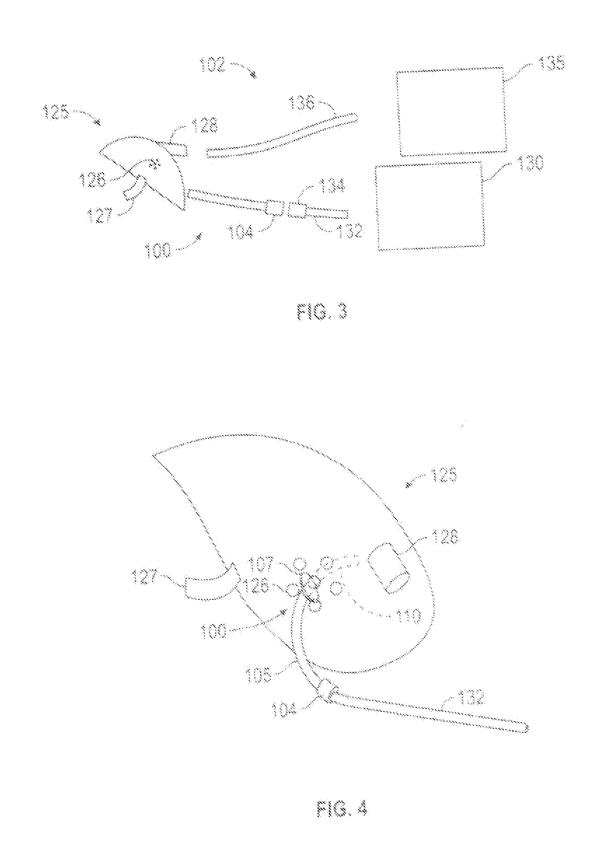

With reference to FIG. 3, a fragmented view of anesthesia monitoring system 102 is illustrated. Oxygen source 135 can be coupled to oxygen connector 128 of mask 125 via oxygen line 136. In various embodiments, mask 125 can be any type that is typically used by those skilled in the art, now or in the future, for medical procedures on either humans or animals. For example, mask 125 can be a Hudson® surgical mask. As illustrated in the Figures, mask 125 comprises oxygen connector 128 which is a port configured to transfer the flow of air to the inside of mask 125. Also as illustrated in the Figures, mask 125 comprises strap 127 configured to hold mask 125 on patient 121. Furthermore as illustrated in the Figures, mask 125 comprises a hole 126. The number of holes 126, the diameter of holes 126, as well as the configuration of holes 126 can vary depending on the supplier of mask 125. In addition the number of holes 126 as well as the configuration of holes 126 can vary depending on size of mask 125. For example, the size of mask 125 may differ between use with adults or with pediatrics, or with infants, or with animals in veterinary applications. In various embodiments, adapter 100 can be interfaced with at least one hole 126.

With reference to FIG. 3, carbon dioxide sample line 132 comprises sample line connector 134. As will be appreciated to those skilled in the art, sample line connector 134 may be designed as a proprietary connector such that only accessories approved by a particular manufacturer of gas analyzer 130 are configured to connect to sample line connector 134. However, various embodiments provide connector 104 comprising any proprietary connector configuration or a reflection thereof, to couple to sample line connector 134. In one embodiment, connector 104 comprises a male connector configured to couple with female connector of sample line connector 134. In one embodiment, connector 104 comprises female connector configured to couple with male connector of sample line connector 134. In one embodiment, connector 104 and sample line connector 134 comprise components of a Luer Lok® connection mechanism. In one embodiment, connector 104 and sample line connector 134 can be coupled with any connector mechanism for a gas-tight coupling of two tubes, including but not limited to any connector mechanism now known to those skilled in the art or is developed in the future. In one embodiment, sample line 132 is integrated into adapter 100 and has connector 104 located at a terminus of sample line 132. In this embodiment, adapter 100 comprises enough length of sample line 132 to allow connector 104 to connector to gas analyzer 130.

Now turning to FIG. 4, adapter 100 coupled to mask 125 is illustrated, in accordance with various embodiments. A portion of adapter 100 ian be placed inside of mask 125. In one embodiment, at least perforated tube 110 and flexible portion 109 are located inside of mask 125 when coupled to adapter 100 (not shown). In various embodiments, adapter 100 can be coupled to mask 125 through one of the holes 126. One embodiment, fitting 107 secures placement of adapter 100 inside of mask 125. Fitting 107 can be coupled between tubing 105 and one of the holes 126. Fitting 107 may be a single piece or a combination of pieces for attachment of tubing 105 to mask 125. In one embodiment, a plurality of different fittings 107 can be incorporated to ensure adapter 100 can be interfaced with a variety of different sizes, shapes, designs, and/or brands of mask 125.

In various embodiments, fitting 107 can be any type of fitting to connect adapter 100 to mask 125 known to those skilled in the art or is developed in the future. In one embodiment, connector 104 is configured to operate as fitting 107 and couple adapter 100 to mask 125. For example, tube 105 is configured to have an outer diameter that is smaller than the diameter of holes 126, which allows tube 105 to be pushed through holes 126 from the inside of mask 125 to mate with connector 104, which is coupled to the exposed end of tube 105, thus coupling tube 105 to mask 125.

In one embodiment, fitting 107 has an annular surface on an end facing towards connector 104 and a diameter of the annular surface is larger than a diameter of one of the holes 126. One embodiment, fitting 107 has at least one of a barbed fitting and a bayonet fitting at an end of fitting 107 facing towards flexible portion 109. For example, perforated tube 110 may be pushed through one of the holes 126 and followed by flexible portion 109 moving through one of the holes 126 until fitting 107 mates with one of the holes 126 coupling adapter 100 to mask 125. In one embodiment, fitting 107 may be pushed into one of the holes 126 allowing the end of fitting 107 facing towards flexible portion 109 to permanently couple adapter 100 mask 125. For example, fitting 107 may be pushed into hole 126 until a barbed fitting or a bayonet fitting clips into place inside of mask 125 thereby coupling adapter 100 to mask 125. In one embodiment, fitting 107 is a tapered portion of tubing 105, which allows for a predetermined length of adapter 100 to be brought into mask 125.

In one embodiment, fitting 107 essentially locks (releasably or permanently) adapter 100 in one of the holes 126 at a certain position within mask 125. As noted, fitting 107 as depicted is one non-limiting example of a size and geometry. As noted above, the fitting 107 can be smaller or larger, and have more than one member to releasably or permanently secure or lock the adapter 100 into a desired position. In one embodiment, fitting 107 increases friction allowing adapter 100 to slide (some amount of force may be applied) into a position within mask 125 while creating enough friction to hold adapter 100 at the position within mask 125. In one embodiment, fitting 107 is permanently fixed to tubing 105, which can provide a repeatable length between fitting 107 and tip 108. In one embodiment, fitting 107 may slide along tubing 105 such that the length between fitting 107 and tip 108 may be adjusted to accommodate a variety of applications, or a variety of mask 125 types, or a variety of mask 125 sizes, or a variety of facial configurations of patient 121. In one embodiment, fitting 107 is both lockable and releasable such that length between fitting 107 and tip 108 may be adjusted to accommodate a variety of applications, or a variety of mask 125 types, or a variety of mask 125 sizes, or a variety of facial configurations of patient 121. In various embodiments, fitting 107 is configured to allow adjustability of tube 105 to place perforated tubing 110 in a tareted area of nasal passage 165, such as, for example, the nasal choana. In one embodiment, tubing 105 can he configured to have predetermined positions to lock or to releasablv lock fitting 107 onto tubing 105. Typically, about 2 to about 4 inches between fitting 107 and tip 108 is a length that is useful for many applications of adapter 100. However, any length between fitting 107 and tip 108 can be used. For example, but not limited to, the length between fitting 107 and tip 108 can be 1 to 3 inches, or 1 to 5 inches, or 2 to 5 inches, or 2 to 6 inches. In some embodiments, tubing 105 comprises graduated marking configured to ensure repeatable positioning of adapter 100 within mask 125. In one embodiment, tubing 105 comprises graduated marking configured for a variety of different sizes, shapes, designs, and/or brands of mask 125 to ensure repeatable and correct positioning of adapter 100 in any of a variety of mask 125. In one embodiment, tithing 105 comprises graduated marktng configured for any of a variety of different sizes, shapes, gender, species, and age groups of patient to position of adapter 100 in a targeted area of nasal passage 165, such as, for example, the nasal choana, in any of a variety of patient 121 types. In some embodiments, fitting 107 may be constructed with multiple pieces. In one embodiment, fitting 107 is integrated with connector 104, into a single piece or a group of multiple pieces.

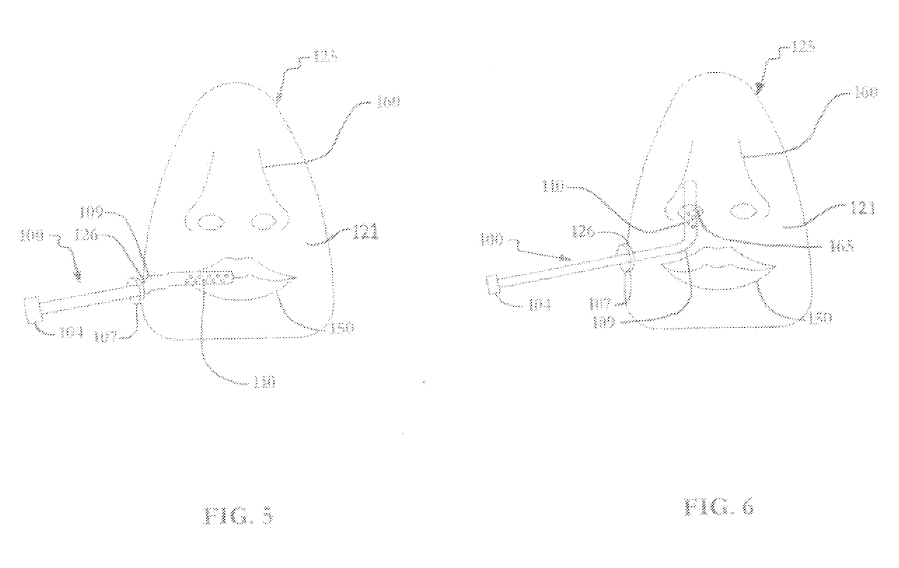

With reference to FIG. 5, a diagrammatic view of mask 125, face of patient 121, and adapter 100 can illustrate a method of use, according to various embodiments. As illustrated, adapter 100 can be coupled to mask 125 through an opening, hole or fitting in mask 125. In various embodiments, a method of use can include coupling adapter 100, having flexible portion 109 connected to perforated tube 110, into hole 126 of mask 125, and placing perforated tube 110 over a portion of oral passage 150. In one embodiment, a method of use can include bending and/or directing flexible portion 109 such that perforated tube 110 is positioned to be in communication with gas exhaled from oral passage 150. In one embodiment, a method of use can include bending and/or directing flexible portion 109 such that perforated tube 110 is positioned to be in communication with gas exhaled from an artificial nasal airway. In one embodiment, a method of use can include bending and/or directing flexible portion 109 such that perforated tube 110 is positioned to be in communication with gas exhaled front an artificial oral airway. In one embodiment, a method of use can include bending and/or directing flexible portion 109 such that perforated tube 110 is positioned to be communication with gas exhaled from at least one of oral passage 150 and nasal passage 165 of patient 121. In one embodiment, a method of use can also include coupling adapter 100 to carbon dioxide sample line 132. In one embodiment, a method of use can also include coupling adapter 100 directly to gas analyzer 130. In one embodiment, a method of use can include collecting gas exhaled by patient 121 from at least one of the oral passage 150 and nasal passage 165 and transfening the gas to gas analyzer 130. In one embodiment, a method of use can include determining an amount of carbon dioxide exhaled by patient 121.

Turning now to FIG. 6, a diagrammatic view of mask 125, face of patient 121, and adapter 100 can illustrate a method of use, according to various embodiments. As illustrated, adapter 100 can be coupled to mask 125. In various embodiments, a method of use can include coupling adapter 100, having flexible portion 109 connected to perforated tube 110, into one of the holes 126 of mask 125, and placing perforated tube 110 into nasal passage 165 or artificial nasal airway in nose 160. In one embodiment, a nasal method of use can include bending and/or directing flexible portion 109 such that perforated tube 110 is positioned to be in communication with gas exhaled from nasal passage 165. In one embodiment, a method of use can include bending and/or directing flexible portion 109 such that perforated tube 110 is positioned to be in communication with gas exhaled from artificial nasal airway. In one embodiment, a method of use can also include coupling adapter 100 to carbon dioxide sample line 132. In one embodiment, a method of use can also include coupling adapter 100 directly to gas analyzer 130. In one embodiment, a method of use can include collecting gas exhaled by patient 121 from nasal passage 165 and transferring the gas to gas analyzer 130. In one embodiment, a method of use can include determining an amount of carbon dioxide exhaled by patient 121. In some embodiments, tubing 105 comprises graduated marking configured to ensure repeatable positioning of adapter 100 within an artificial nasal airway. In one embodiment, tubing 105 comprises graduated marking configured for a variety of different sizes, shapes, gender, species, and age groups of patient to ensure repeatable and correct positioning of adapter 100 in any of a variety of patient 121 types. In one embodiment, tubing 105 comprises graduated marking configured for a variety of different sizes, shapes, gender, species, and age groups of patient to position of adapter 100 in a targeted area of nasal passage 165, such as, for example, the nasal choana, in any of a variety of patient 121 types.

In one embodiment, nasal passage 165 in nose 160 can be a nasopharynx with the artificial nasal airway in place. In one embodiment, nasal passage 165 in nose 160 can be a aasopharynx when the artificial nasal airway is inserted alone deep into the nasopharyngeal area/space. In one embodiment, nasal passage 165 in nose 160 can be the nare or an edge of the nare. In various embodiments, when adapter 100 is employed for monitoring an artificial nasal airway, adapter 100 can be placed deep in a nasal cavity for nasopharyngeal airway sampling and/or monitoring. In various embodiments, adapter 100 can be positioned in an artificial nasal airway and then adjusted to detect carbon dioxide in any nasal-pharynx alone or within a nasopharvugeal airway.

Moving to FIG. 7, a diagrammatic view of mask 125, face of patient 121, and adapter 100 can illustrate a method of use, according to various embodiments. In various embodiments, a method of use can include coupling adapter 100, having flexible portion 109 connected to perforated tube 110, into hole 126 of mask 125, and placing perforated tube 110 into, across, or near oral passage 150, such as, for example a mouth. In one embodiment, a method of use can include bending and/or directing flexible portion 109 such that perforated tube 110 is positioned to be in communication with gas exhaled from oral passage 150. hi one embodiment, a method of use can include bending and/or directing flexible portion 109 such that perforated tube 110 is positioned inside of artificial oral airway 168. In one embodiment, a method of use can include placing perforated tube 110 into artificial oral airway 168, such as for example a tube for direct oropharynx detection of carbon dioxide. In one embodiment, a method of use can also include coupling adapter 100 directly to gas analyzer 130. In one enibodiment, a method of use can include collecting gas exhaled by patient from inside artificial oral airway 168 and transferring the gas to gas analyzer 130. In one embodiment, a method of use can include determining an amount of carbon dioxide exhaled by patient 121. In various embodimentd, when adapter 100 is employed for monitoring an oral passageway adapter 100 can be placed deep in artificial oral airway 168 for oropharyngeal airway sampling and/or monitoring.

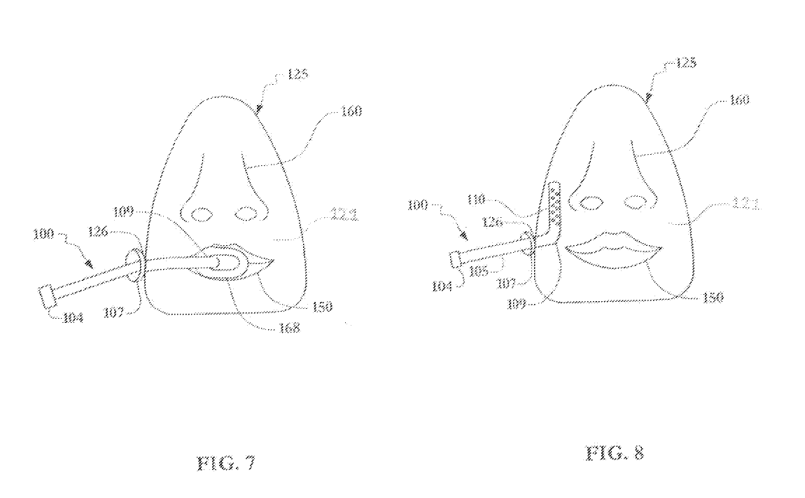

In FIG. 8, a diagammatic view of mask 125, face of patient 121, and adapter 100 can illustrate a method of use, according to various embodiments. In various embodiments, a method of use can include coupling adapter 100, having flexible portion 109 connected to perforated tube 110, into hole 126 of mask 125, and positioning perforated tube 110 inside mask 125. One embodiment, the method can include bending and/or directing flexible portion 109 such that perforated tube 110 is in communication with gas exhaled by patient 121. In one embodiment, a method of use can also include coupling adapter 100 directly to gas analyzer 130. In one embodiment, a method of use can include collecting the gas exhaled by patient 121 and transferring the gas to gas analyzer 130. In one embodiment, a method of use can also include determining an amount of carbon dioxide exhaled by patient 121.

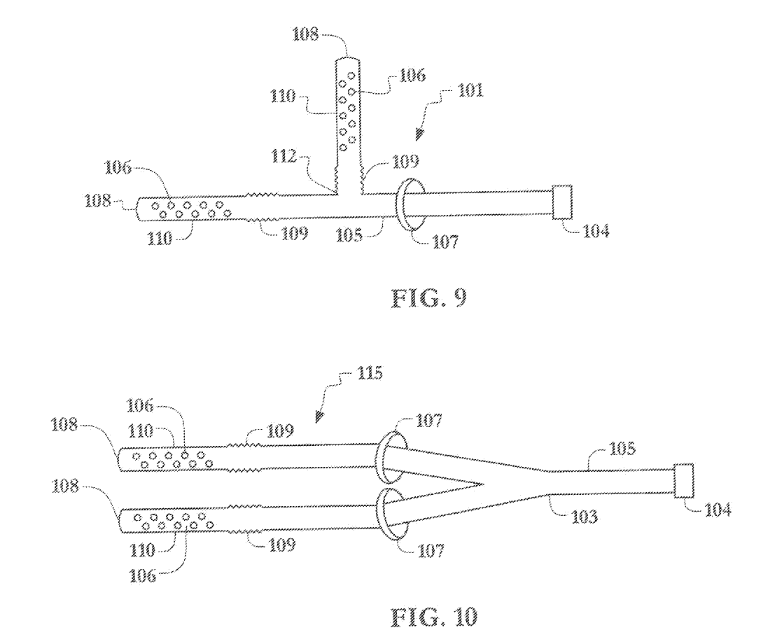

Now with reference to FIG. 9, adapter 101 comprising a plurality of perforated tubes 110 and fitting 107 is illustrated, according to various embodiments. Accordingly, in one embodiment, adapter 101 can be used to monitor gas exhaled by patient 121 in more than one location within mask 125. Adapter 101 can be configured for duel catheter detection. For adapter 101 may be positioned to be communication with gas exhaled from patient 121 from both oral passage 150 and nasal passage 165. In another example, adapter 101 may be positioned to be in communication with Ras exhaled from patient 121 from oral passage 150 and another location within mask 125. In still another example, adapter 101 maybe positioned to be in communication with gas exhaled from patient 121 from nasal passage 165 and another location within mask 125. As will be apparent to those skilled in the art, another location within the mask 125 can be a location, for example, as illustrated in FIG. 8 and described herein. In one embodiment, adapter 101 comprises a plurality of perforated tubes 110 and the flexible portion 109 connected to each of the plurality of perforated tubes 110. In one embodiment, adapter 101 comprises a plurality of perforated tubes 110 and one flexible portion 109 connected to one of the plurality of perforated tubes 110. For example, adapter 101 can comprise one perforated tube 110 coupled to flexible portion 109 for movably positioning perforated tube 110 at any location within mask 125 and can comprise another perforated tube 110 coupled to tubing 105 which may be placed in communication with oral passage 150. In one embodiment, adapter 101 is configured for placement of one perforated tube 110 into nasal passage 165 in nose 160 and another for placement in an area above oral passage 150, such as, for example a mouth. In some embodiments, adapter 101 can be coupled directly to gas analyzer 130.

Similarly, although adapter 101 is shown with two perforated tubes 110, each with a flexible portion 109, and each with a plurality of holes 106, it should be understood that in some cases, one tube can have a single hole 106 while the other has multiple andior one perforated tube 110 can have the flexible portion 109 while the other does not, etc. Also, while not shown, each perforated tube 110 can feed into a single chamber with in tube 105 or into separate chambers or passageways within tube 105. As such, gases from the different locations can be separately analyzed and compared, if desired.

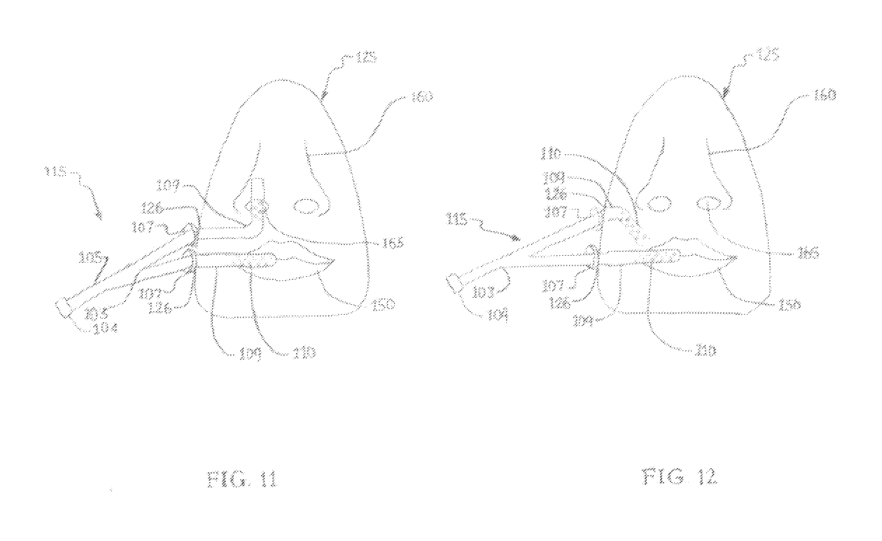

With attention directed to FIG. 10, adapter 115 comprising a plurality of perforated tubes 110 and a plurality of fittings 107 is illustrated, according to various embodiments. Apparatus 115 can comprise connector 104, such as described herein, configured to detachably connect to carbon dioxide sample line 132. As illustrated, connector 104 can be coupled to tubing 105. In one embodiment, connector 104 and tubing 105 are separate components with connector 104 configured to be seated around tubing 105. In one embodiment, connector 104 is fused to or is integral to tubing 105. Also as illustrated in the Figures, tubing 105 can comprise manifold 103, such as for a Y in tubing 105. In various embodiments, each fitting 107 is located between manifold 103 and tip 108 and is configured to interface with mask 125, as described herein. At ends of each of a plurality of tubing 105 distal to manifold 103 is flexible portion 109. Adapter 115 can be configured for duel catheter detection. In one embodiment, adapter 115 can be connected directly to gas analyzer 130. In one embodiment, flexible portion 109 can be moved in any direction. In various ethbodiments, flexible portion 109 can be constructed from a material which is both flexible and has shape memory. In one embodiment, flexible portion 109 comprises enough elasticity to bend into a position and enough rigidity to maintain the position over a period of time.

In various embodiments, as illustrated in the Figures, each of a plurality of flexible portion 109 is coupled to one of the plurality of perforated tube 110. In various embodiments, the plurality of perforated tube 110 is in communication through adapter 115 to gas analyzer 130. In one embodiment, the plurality of perforated tube 110 is configured to transfer carbon dioxide exhaled by patient 121 to gas analyzer 130.

As illustrated in FIG. 11, adapter 115 is coupled or secured to mask 125 in more than one location. A portion of adapter 115 can be placed inside of mask 125. In one embodiment, at least a plurality of perforated tube 110 and a plurality of flexible portion 109 are located inside of mask 125 when coupled to adapter 115. In various embodiments, adapter 115 can be coupled to mask 125 through hole 126. In one embodiment, at least one fitting 107 secures placement of adapter 115 inside of mask 125. Fitting 107 can be coupled to mask 125 between manifold 103 and hole 126. In one embodiment, fitting 107 has an annular surface facing towards manifold 103 and a diameter of the annular surface is larger than a diameter of hole 126. In one embodiment, fitting 107 has at least one of a barbed fitting and a bayonet fitting at in end of fitting 107 facing towards tip 108. Other fitting orientations and geometries can be utilized, as well, as discussed herein.

For example, each of the plurality of perforated tube 110 may be pushed through hole 126 and followed by flexible portion 109 moving through the hole 126 until at least one fitting 107 mates within one hole 126 thereby coupling adapter 115 to mask 125. In one embodiment, at least one fitting 107 may be pushed into one hole 126 allowing the end of the at least one fitting 107 facing towards tip 108 to permanently couple adapter 115 mask 125. For example, the at least one fitting 107 may be pushed into the hole 126 until a barbed fitting or a bayonet fitting clips into place inside of mask 125 thereby coupling adapter 115 to mask 125. Although FIG. 10 illustrates a plurality of perforated tube as being 2, any number of perforated tubes 110 may be employed, in accordance to various embodiments. For example, a plurality of perforated tube 110 can be 3 such that one of the plurality of perforated tube 110 can be placed in or around oral passage 150, such as, for example, a mouth, and another of the plurality of perforated tube 110 can be placed in, or across, or near an nasal passage 165 in nose 160, and still another of the plurality of perforated tube 110 can be placed in a position within mask 125.

In one embodiment,at least one fitting 107 essentially locks adapter 115 in one hole 126 at a certain position within mask 125. In one embodiment, fitting 107 increases friction allowing each of the plurality of perforated tubes 110 to slide into a position within mask 125 while creating enough friction to hold the each of the plurality of perforated tubes 110 at the position within mask 125. As discussed herein, fitting 107 can be any type of fitting to connect adapter 115 to mask 125 blown to those skilled in the art or is developed in the future. In one embodiment, connector 104 is configured to operate as fitting 107. For example, tube 105 is configured to have an outer diameter that is smaller than the diameter of hole 126, which allows tube 105 to be pushed through hole 126 from the inside of mask 125 to mate with connector 104, which is coupled to the exposed end of tube 105, thus coupling tube 105 to mask 125. In one embodiment, connector 104 can also function as fitting 107. In some embodiments, connector 104 has an outer diameter that is smaller than the diameter of hole 126, which allows connector 104 to be pushed through hole 126 from the inside of mask 125 to mate with sample line 132. In this embodiment, connector 134 may operate as fitting 107 or as a portion of fitting 107.

The use of multiple fittings 107, including fittings that permit securement into more than one position for each perforated tube 110 can permit each perforated tale 110 to have a desired length and positioning with respect to the patient 121. For example, one can be secured “longer” so that one perforated tube 110 can be positioned deep into the oropharyngeal airway or nasal passageway, while the other perforated tube 110 is secured “shorter” so that it can be positioned outside of the nasal passage 165 or over the oral passage 150, for example. Fittings 107 with bumps or protrusions can be used, for example, in such cases, or any other suitable orientation can be used.

In some embodiments, tubing 105 comprises graduated marking configured to ensure repeatable positioning of adapter 100 within mask 125. In one embodiment, tubing 105 comprises graduated marking configured for a variety of different sizes, shapes, designs, and/or brands of mask 125 to ensure repeatable and correct positioning of adapter 100 in any of a variety of masks 125. In one embodiment, tubing 105 comprises graduated marking configured for any of a variety of different sizes, shapes, gender, species, and age groups of patient to position of adapter 100 in a targeted area of nasal passage 165, such as, for example, the nasal choana, in any of a variety of patient 121 types.

Various embodiments, adapter 115 can be used to monitor gas exhaled by patient 121 in more than one location within mask 125. For example, adapter 115 may be positioned to be in communication with gas exhaled from patient 121 from both oral passage 150 and nasal passage 165. In another example, adapter 115 may be positioned to be in communication with gas exhaled from patient 121 from oral passage 150 and another location within mask 125. In still another example, adapter 115 may be positioned to be in communication with gas exhaled from patient 121 from nose 160 and another location within mask 125. As will be apparent to those skilled in the art, another location within the mask 125 can be a location as illustrated in FIG. 8 and described herein.

Again referring to FIG. 11, a diagrammatic view of mask 125, face of patient 121, and adapter 115 can illustrate a method of use, according to various embodiments. As illustrated, adapter 115 can be coupled to mask 125. In various embodiments, a method of use can include coupling adapter 115, having a plurality of flexible portions 109, each connected to one of a plurality of perforated tubes 110, into hole 126 of mask 125, and positioning one of the plurality of perforated tube 110 in or near a portion of oral passage 150 and positioning another of the plurality of perforated tubes 110 in or near a nasal passage 165 in nose 160. In one embodiment, a method of use can include positioning one of the plurality of perforated tubes 110 into a certain location within mask 125. In one embodiment, a method of use can include bending and/or directing flexible portion 109 such that perforated tubes 110 is positioned to be in communication with gas exhaled from patient 121 in at least two locations. In one embodiment, a method of use can include bending, and/or directing a plurality of flexible portions 109 such that one of the plurality of perforated tubes 110 is positioned to be in communication with gas exhaled from oral passage 150 and another of the plurality of perforated tubes 110 is positioned to be in communication with gas exhaled from nasal passage 165. The method of use can include configuring adapter 115 for duel catheter detection.

In one embodiment a method of use on also include coupling adapter 115 to carbon dioxide sample line 132. In one embodiment, a method of use can also include coupling adapter 115 directly to gas analyzer 130. In one embodiment, a method of use can include collecting gas exhaled by patient 121 and transferring the gas to gas analyzer 130. In one embodiment, a method of use can include determining an amount of carbon dioxide exhaled by patient 121. In various embodiments, adapter 115 can be employed for both nasal passage sampling and/or monitoring and oral passage sampling and/or monitoring. In one embodiment, adapter 115 can be employed for both nasopharyngeal sampling and/or monitoring and oropharyngeal sampling andlor monitoring.

Moving to FIG. 12, a diagrammatic view of mask 125, face of patient 121, and adapter 115 can illustrate a method of use, according to various embodiments. As illustrated, adapter 115 can be coupled to mask 125. In various embodiments, a method of use can include coupling adapter 115, having a plurality of flexible portions 109, each connected to one of a plurality of perforated tubes 110, into hole 126 of mask 125 or through connector 104, and positioning one of the plurality of perforated tubes 110 in or near a portion of oral passage 150 and positioning another of the plurality of perforated tubes 110 in a predetermined location within mask 125. In one embodiment, a method of use can include bending and/or directing a plurality of flexible portions 109 such that one of the plurality of perforated tubes 110 is positioned to be in communication with gas exhaled from oral passage 150 and another of the plurality of perforated tithes 110 is positioned to be in communication with gas exhaled at a predetermined position within mask 125. In one embodiment a method of use can also include coupling adapter 115 to carbon dioxide sample line 132. In one embodiment, a method of use can include collecting gas exhaled by patient 121 transferring the gas to gas analyzer 130. In one embodiment, a method of use can include determining an amount of carbon dioxide exhaled by patient 121.

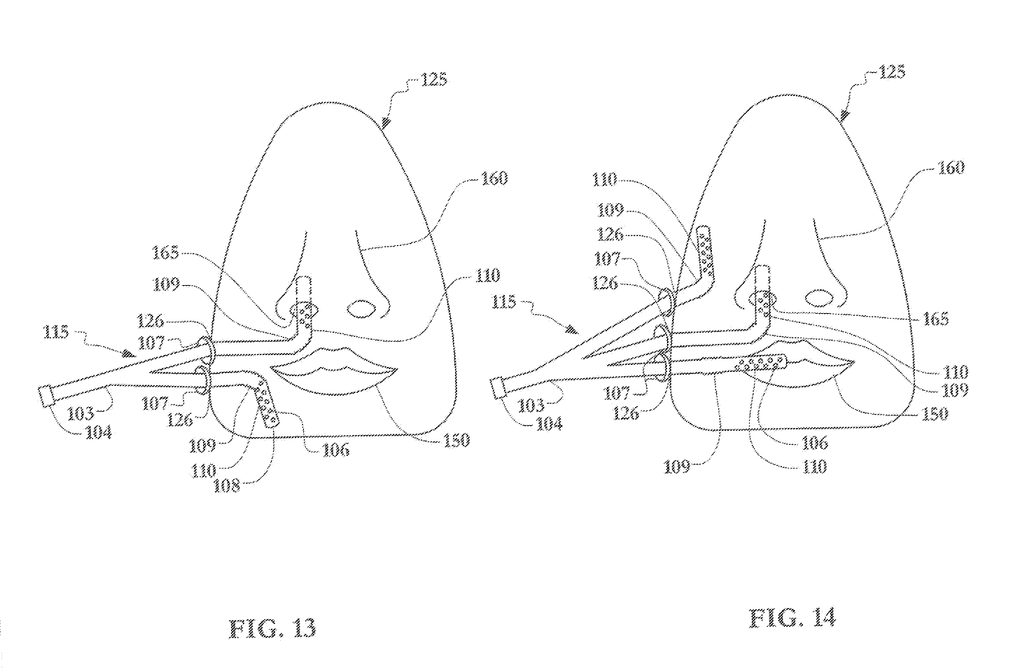

Turning to FIG. 13, a diagrammatic view of mask 125, face of patient 121, and adapter 115 can illustrate a method of use, according to various embodiments. As illustrated, adapter 115 can be coupled to mask 125. In various embodiments, a method of use can include coupling adapter 115, having a plurality of flexible portions 109, each connected to one of a plurality of perforated tubes 110, into hole 126 of mask 125, and positioning one of the plurality of perforated tubes 110 in or near a nasal passage 165 in nose 160 and positioning another of the plurality of perforated tubes 110 in a predetermined location within mask 125. In one embodiment, a method of use can include bending and/or directing a plurality of flexible portions 109 such that one of the plurality of perforated tubes 110 is positioned to be in communication with gas exhaled from nasal passage 165 and another of the plurality of perforated tubes 110 is positioned to be in communication with gas exhaled at a predetermined position within mask 125. In one embodiment a method of use can also include coupling adapter 115 to carbon dioxide sample line 132. In one embodiment, a method of use can also include coupling adapter 115 directly to gas analyzer 130 in one embodiment, a method of use can include collecting has exhaled by patient 121 transferring the gas to gas analyzer 130. In one embodiment, a method of use can include determining an amount of carbon dioxide exhaled by patient 121.

Finally with reference to FIG. 14, a diaammatic view of mask 125, face of patient 121, and adapter 115 can illustrate a method of use, according to various embodiments. As illustrated, adapter 115 can be coupled to mask 125. In various embodiments, a method of use can include coupling adapter 115, having a plurality of flexible portions 109, each connected to one of a plurality of perforated tubes 110, into a hole 126 of mask 125, and positioning one of the plurality of perforated tube 110 in or near a portion of oral passage 150 and positioning another of the plurality of perforated tubes in or near an nasal passage 165 in nose 160 and positioning one of the plurality of perforated tube 110 into a certain location within mask 125. In one embodiment, a method of use can include bending and/or directing, flexible portion 109 such that perforated tube 110 is positioned to be in communication with gas exhaled from patient 121 in at least three locations. In one embodiment, a method of use can include bending and/or directing a plurality of flexible portions 109 such that one of the plurality of perforated tubes 110 is positioned to be communication with gas exhaled from oral passage 150, one of the plurality of perforated tubes 110 is positioned to be communication with gas exhaled from nasal passage 165 in nose 160, and another of the plurality of perforated tubes 110 is positioned to be communication with ambient air in mask 125 containing gas exhaled from nasal passage 165. In one embodiment a method of use can also include coupling adapter 115 to carbon dioxide sample line 132. In one embodiment, a method of use can also include coupling adapter 115 directly to gas analyzer 130. In one embodiment, a method of use can include collecting gas exhaled by patient 121 transferring the gas to gas analyzer 130. In one embodiment, a method of use can include determining an amount of carbon dioxide exhaled by patient 121.

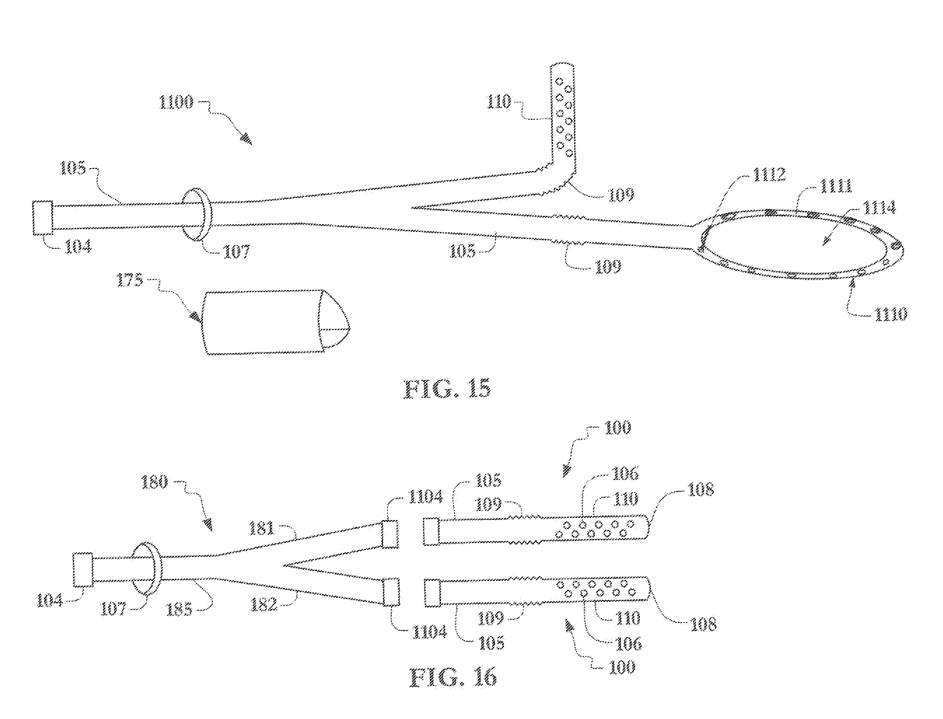

Now moving to FIG. 15, adapter 1100 comprising a non-limiting example of a mouthpiece 1110 is illustrated, according to various embodiments. Adapter 1100 comprises connector 104 configured to detachably connect to carbon dioxide sample line 132. In one embodiment, connector 104 comprises a male connector configured to couple with a female connector on carbon dioxide sample line 132. In one embodiment, connector 104 comprises a female connector configured to couple with a male connector on carbon dioxide sample line 132. In one embodiment, connector 104 comprises a Luer Lok® connector, which is well known to those skilled in the art. In various embodiments, connector 104 can be configured to interface or couple to any connector on carbon dioxide sample line 132 or gas analyzer.

In various embodiments, connector 104 is coupled to tubing 105. Tn one embodiment, connector 104 and tubing 105 are separate components with connector 104 configured to be seated around tubing 105. In one embodiment, connector 104 is fused to tubing 105. Also as illustrated in the Figures, tubing 105 interfaces with fitting 107. In various embodiments, fitting 107 is configured to interface with mask 125, as described herein. However, in some embodiments, adapter 1100 does not include fitting 107. In one embodiment, connector 104 and fitting 107 are integrated into one piece, which operates both as the fitting and as the connector. At an end of tubing 105 distal to connector 104 is flexible portion 109. In one embodiment, flexible portion 109 can be moved in any direction, as discussed herein. In some embodiments, flexible portion 109 is constructed from a material which is flexible and can have enough elasticity to be bent into a position. For example, such a material can be flexible enough to bend but not crimp flexible portion 109 and in some examples such a material may be able to keep the shape of the bend in flexible portion 109 for a period of time.

In some embodiments, flexible portion 109 is constructed from a material which is both flexible and has shape memory. In one embodiment, flexible portion 109 comprises enough elasticity to be bent into a position and enough rigidity to maintain the position over a period of time. For example, such a material can be flexible enough to bend but not crimp flexible portion 109 and should be able to keep the shape of the bend in flexible portion 109 for a period of time, as discussed herein. In one embodiment, tubing 105 and/or flexible portion 109 can be configured to absorb water in the inteinal surface of tubing 105 and/or flexible portion 109.

In various embodiments, as illustrated in FIG. 15, flexible portion 109 is coupled to perforated tube 110. In one embodiment, flexible portion 109 and perforated tube 110 are separate components, which are at least one of mechanically, physically, and chemically attached to one another. In one embodiment, flexible portion 109 and perforated tube 110 are fused together as a continual piece. Perforated tube 110 comprises a plurality of holes 106 which are in communication through adapter 1100 to gas analyzer 130. In one embodiment, plurality of holes 106 can be a plurality of pores in a membrane which is coupled to a portion of perforated tube 110.

In one embodiment, perforated tube 110 comprises tip 108, which can be hollow. In one embodiment, tip 108 is in communication through adapter 1100 to gas analyzer 130. Various embodiments, at least one of plurality of holes 106 and tip 108 is configured to receive a gas exhaled by the patient 121, which can be sampled by gas analyzer 130. In one embodiment, at least one of plurality of holes 106 and tip 108 is configured to transfer carbon dioxide exhaled by patient 121 to gas analyzer 130. In one embodiment, tip 108 is closed or is solid. As discussed herein, tip 108 can comprise at least one of soft edges, rounded edges, and chamfered edges, which can minimize damage to tissue as adapter 1100 is placed in an airway. In one embodiment, at least one tip 108 and perforated tube 110 is weighted, which in at least one of placement of adapter 1100 in at airway, and keeping adapter 1100 positioned in an airway while patient 121 is breathing. In one embodiment, adapter 1100 can comprise a balloon coupled to a portion of adapter 1100 and configured to secure a portion of adapter 1100 in an airway of patient 121. In one embodiment, adapter 1100 can comprise a weighted member coupled to a portion of adapter 1100 and configured to secure a position of adapter 1100 in an airway of patient 121.

In various embodiments, adaptor 1100 comprises mouthpiece 1110. In some embodiments, mouthpiece 1110 comprises edge 1111, which may be formed to fit over the contour around oral passage 150, such as, for example, mouth, lips, and/or surrounding skin. In some embodiments, adapter 1100 can be configured in a spoon-like shape comprising a plurality of openings 1112 in communication with tubing 105. In one embodiment, adapter 1100 can be configured with a plurality of openings 1112 along inner edge 1111 of the spoon-like shape. In one embodiment, edge 1111 comprises a removable adhesive material to fasten mouthpiece 1110 over oral passage 150, such as, for example a mouth. In one embodiment, edge 1111 comprises sticky material to fasten mouthpiece 1110 over oral passage 150, such as, for example a mouth. Mouthpiece 1110 comprises a plurality of openings 1112 which is in fluid communication with tubing 105. In one embodiment, a portion of flexible portion 109 can be between tubing 105 and mouthpiece 1110. The flexible portion 109, as described herein, facilitates the positioning of mouthpiece 1110 over oral passage 150, such as, for example a mouth.

In various embodiments described herein, any perforated tube 110 can be replaced with mouthpiece 1110. For example, any embodiment comprising perforated tube 110 can comprise mouthpiece 1110 instead of perforated tube 110. In some embodiments, center area 1114 located inside of edge 1111 of mouthpiece 1110 can comprise a perforated film or perforated layer, which is in fluid communication with opening 1112 (not shown). In some embodiments, center area 1114 can have a concave shape. In some embodiments, center area 1114 can be configured in a cup-like shape. In one embodiment, the perforated film or perorated layer may further comprise a filter, which is either integrated thereto or attached thereto. In one embodiment, at least a portion of adapter 1100 is configured to absorb water in the internal surface, such as, for example, Nafion® tubing, which is configured to absorb water in the internal surface of the tubing. In one embodiment, a portion of mouthpiece 1110 is configured to absorb water in the internal surface, such as, for example, center portion 1114 and/or edge 1111.

In some embodiments, adapter 100 or adapter 1100 can be affixed to an oxygen-supply nasal cannula with fastener 175. Fastener 175 can be, for example, a clip, a clamp, an adhesive strip, a hook and loop connector, a vise, bracket clasp, snap, connector, link, tie, or combinations thereof. Fastener 175 should not crimp adapter 100 or adapter 1100, which thus can limit or eliminate flow to gas analyzer 130. Fastener 175 can be removable. In some embodiments, fastener 175 can fix adapter 100 or adapter 1100 to mask 125 for a one time use (for example, not removable). In some embodiments, for adjustability fastener 175 can movably fix adapter 100 or adapter 1100 for either mask or cannula applications. In one embodiment, adapter 1100 can be integrated into or onto an oxygen-supply nasal cannula.

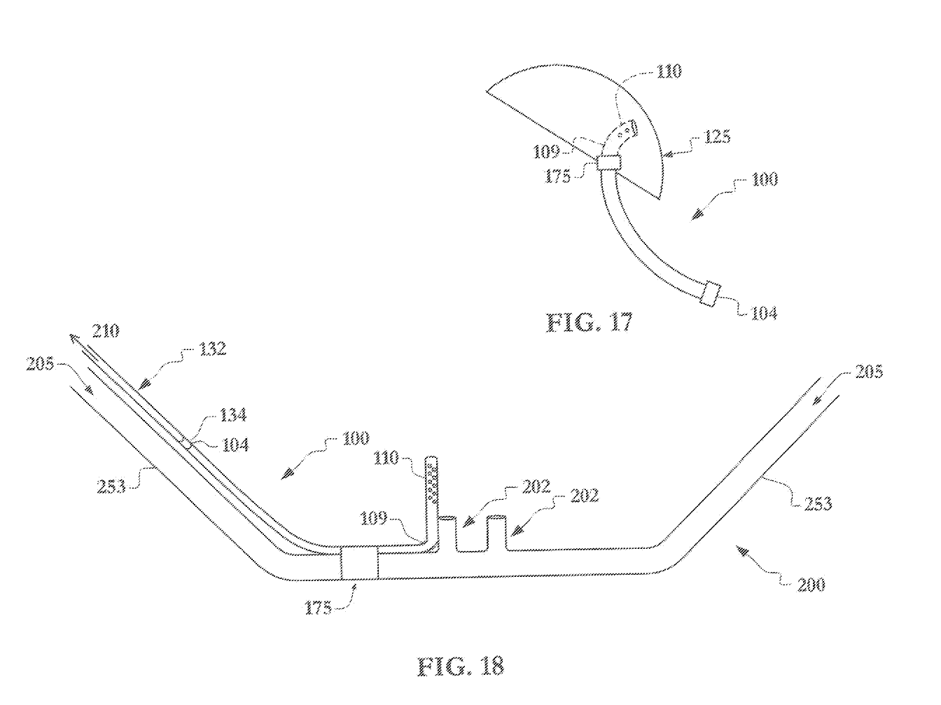

Turning to FIG. 16 is a fragmented view illustrating an adapter 100, according to various embodiments. Y-connecter 180 comprises tubing 185, which is equivalent to tubing 105 described herein. Y-connector 180 comprises first split tubing 181 and second split tubing 182, which are both coupled to Y-connector and equivalent to tubing 105 described herein. Each of first split tubing 181 and second split tubing 182 comprises connector 1104 at the end distal to tubing 185. In some embodiments, connector 1104 is a female connector configured to couple with connector 104 or any other type of connector, as described herein.

Y-connector 180 can be coupled to one or more of adaptor 100. Y-connector 180 can be coupled to one or more of adaptor 1100. Y-connector 180 can be coupled to a combination one or more of adaptor 100 and one or more adapter 1100. In some embodiments, Y-connector 180 comprises three different split tribings. In some embodiments, Y-connector 180 comprises four or more different split tubings. In some embodiments, at least one or more of adapter 100 and adapter 1100 can be permanently attached to Y-connector 180. In one embodiment, tubing 185 can be threaded through hole 126 in mask 125. In one embochment, fitting 107 can lock Y-cormecfor to mask 125. In some embodiments, at least one or more of adapter 100 and adapter 1100 can be connected to one or more connectors 1104, which are located within mask 125. In one embodiment, Y-connector 180 allows a practitioner to thread only one tube through mask 125 instead of multiple tubes through mask 125 when at least one or more of adapter 100 and adapter 1100 are employed.

However, in some embodiments, fitting 107 is not included with Y-connector 180. In such embodiments, connector 1104 can be configured to operate as both a connector and as a fitting. In such embodiments, mask 125 can be positioned between connector 1104 and Y-connector 180 for coupling adapter 100 to mask 125.

In some embodiments, adapter 100 does not comprise fitting 107. In various embodiments, adapter 100 can be placed between mask 125 and skin, as illustrated in, for example, FIG. 17. This is especially advantageous when mask 125 does not comprise hole 126. In various embodiments, adapter 100 can be fixed to mask 125 with fastener 175. For example, fastener 175 may comprise, but is not limited to, a clip, a clamp, an adhesive strip, a hook and loop connector, a vise, bracket, clasp, snap, connector, link or combinations thereof. Fastener 175 should not crimp adapter 100, which thus can limit or eliminate flow to gas analyzer 130. Fastener 175 can be removable. In some embodiments, fastener 175 can fix adapter 100 to mask 125 for a one time use (for example, not removable). In some embodiments, for adjustability fastener 175 can movably fix adapter 100 for either mask or cannula applications.

With reference to FIG. 18, oxygen supply 200 and adapter 100 affixed thereto with fastener 175 is illustrated, according to various embodiments. In some embodiment, fitting 107 is not included with adapter 100. Oxygen supply 200 can include one or more oxygen cannula 202, which may be configured for insertion into a patient's 121 nasal passage 165. Oxygen 205 flows from source (not illustrated) through oxygen supply tube 253 and exits through cannula 202 to supply oxygen 205 to patient 121 through nasal passage 165. In some embodiments, oxygen supply 200 can be any type of nasal cannula which are well known to those skilled in the art. In various embodiments, perforated tube 110 extends into nasal passage 165 and is configured to be positioned above and beyond the top of the cannula 202. In various embodiments, adapter 100 can be fixed to oxygen supply 200 with fastener 175. In one embodiment, adapter 100 can be configured to be connected directly to gas analyzer 130.

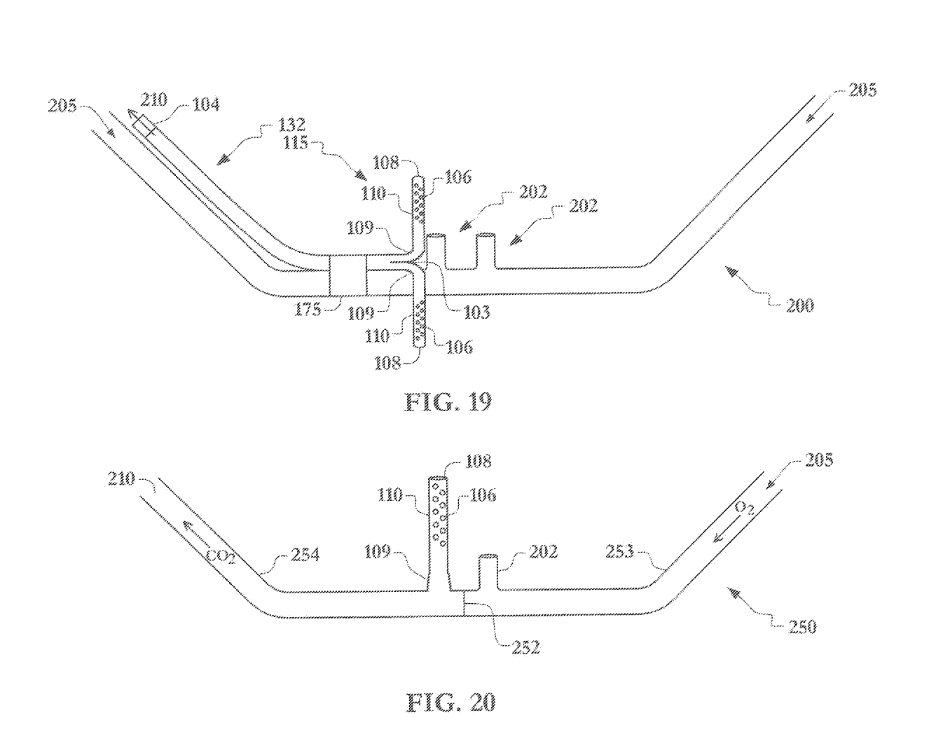

Referring to FIG. 19, oxygen supply 200 and adapter 115 affixed thereto with fastener 175 is illustrated. In some embodiments, fitting 107 is not included with adapter 115. In various embodiments, adapter 115 comprises a plurality of perforated tubes 110 (with reference, for example, to FIG. 10). Adapter 115 can comprise connector 104, such as described herein, configured to detachably connect to carbon dioxide sample line 132 or directly to gas analyzer 130. As illustrated, connector 104 can be coupled to tubing 105. In one embodiment, connector 104 and tubing 105 are separate components with connector 104 configured to be seated around tubing 105. In one embodiment, connector 104 is fused to or is integral to tubing 105. Also as illustrated herein, tubing 105 can comprise manifold 103, such as for a Y in tubing 105. At ends of each of a plurality of tubing 105 distal to manifold 103 is flexible portion 109, as described herein. Each of the plurality of flexible portion 109 is coupled to one of the plurality of perforated tube 110. In various embodiments, the plurality of perforated tube 110 is in communication through adapter 115 to gas analyzer 130. In various embodiments, adapter 115 can be fixed to oxygen supply 200 with fastener 175.

In some embodiments, one of the plurality of perforated tube 110 is positioned in nasal passage 165 and the another of the plurality of perforated tube 110 is positioned over oral passage 150. In one embodiment, one of the plurality of perforated tribe 110 is replaced by mouthpiece 1110. In accordance with this embodiment, one of the plurality of perforated tube 110 is positioned in nasal passage 165 and mouthpiece 1110 is positioned over oral passage 150, such as, for example a mouth. In some embodiments, perforated tube 110 extends into nasal passage 165 and is configured to be positioned above and beyond the top of the cannula 202.

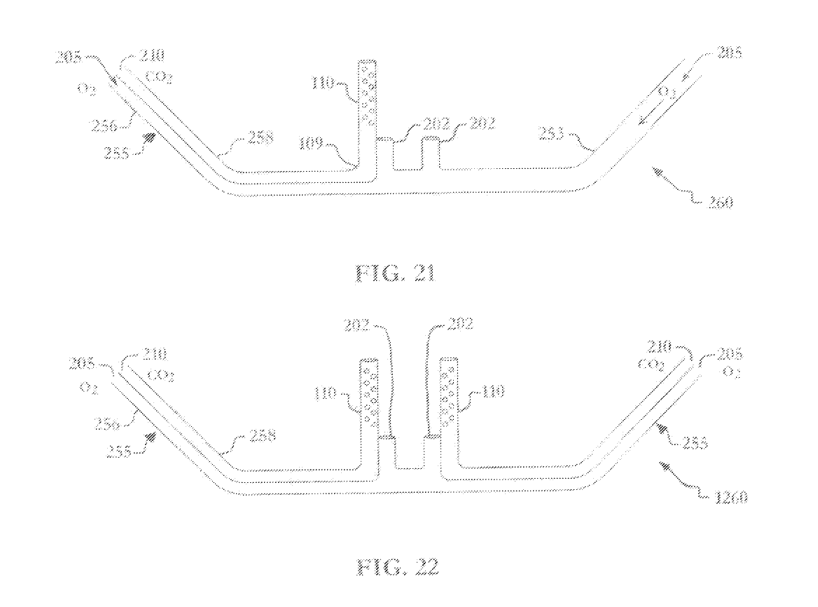

Now moving to FIG. 20, combination device 250 is illustrated. In various embodiments, combination device 250 comprises oxygen supply tube 253, expiration tube 254, at least one cannula 202, at least a portion of adapter 100, and wall 252. In some embodiments, the at least a portion of adapter 100 includes perforated tube 110 and flexible portion 109. In some embodiments, perforated tube 110 extends into nasal passage 165 and is configured to be positioned above the top of the cannula 202. In some embodiments, the flexible portion is coupled to expiration tube 254. Oxygen 205 flows from source (not illustrated) through oxygen supply tube 253 and exiting through cannula 202 to supply oxygen 205 to patient 121 through nasal passage 165. In various embodiments, the at least a portion of adapter 100 is coupled to expiration 254. Carbon dioxide 210 is released by patient and flows from the at least a portion of adapter 100 positioned in nasal passage 165 and through expiration 254, which may be in communication with gas analyzer 130. In various embodiments, wall 252 provides a barrier between oxygen supply tube 255 and expiration tube 254 and is configured for separation of oxygen 205 and carbon dioxide 210.

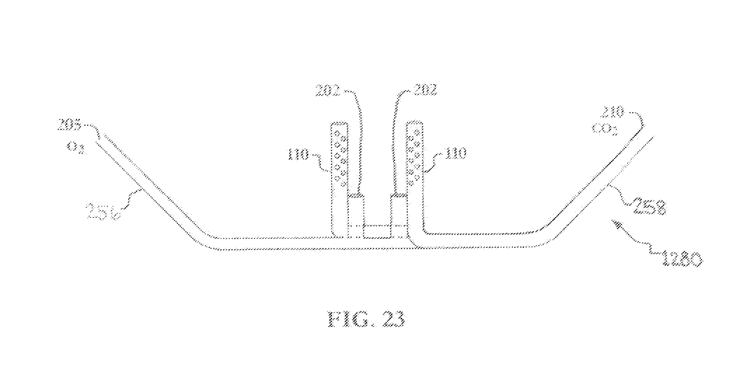

With reference to FIG. 21, combination device 260 is illustrated. In various embodiments, combination device 260 comprises oxygen supply tube 253, combination tube 255, at least one cannula 202, at least a portion of adapter 100. In some embodiments, the at least a portion of adapter 100 includes perforated tube 110 and flexible portion 109. In some embodiments, perforated tube 110 extends into nasal passage 165 and is configured to be positioned above and beyond the top of the cannula 202. In some embodiments of combination device 260, the at least a portion of adapter 100 includes perforated tube 110 coupled to combination tube 255. In one embodiment of combination device 260, the at least a portion of adapter 100 includes perforated tube 110 coupled to combination tube 255 and attached to one of the at least one caunnla 202. In various embodiments, perforated tube 110 is configured to extend into nasal passage 165 and is configured to be positioned above and beyond the top of the cannula 202.

In various embodiments, combination tube 255 comprises oxygen portion 256 and carbon dioxide portion 258. Oxygen 205 flows from source (not illustrated) through oxygen supply tube 253 and exiting through camula 202 to supply oxygen 205 to patient 121 through nasal passage 165. In addition, oxygen 205 flows from source (not illustrated) through oxygen portion 256 and exiting through cannula 202 to supply oxygen 205 to patient 121 through nasal passage 165. In various embodiments, the at least a portion of adapter 100 is coupled to carbon dioxide portion 258. Carbon dioxide 210 is exhaled by patient 121 and flows from the at least a portion of adapter 100 through carbon dioxide portion 258, which may be in communication with gas analyzer 130.

With reference to FIG. 22, combination device 1260 comprises more than one combination tube 255, than one cannula 102 and more than one of the at least a portion of adapter 100. In some embodiments, the at least a portion of adapter 100 includes perforaed tube 110 and flexible portion 109. In some embodiments, perforated tube 110 extends into nasal passage 165 and is configured to be positioned above and beyond the top of the cannula 202. In some embodiments of combination device 1260, the at least a portion of adapter 100 includes perforated tube 110 coupled to combination tube 255. In one embodiment of combination device 1260. the at least a portion of adapter 100 includes perforated tube 110 coupled to combination tube 255 and attached to one of the at least one cannula 202. In various embodiments, combination tube 255 comprises oxygen portion 256 and carbon dioxide portion 258. Oxygen 205 flows from source (not illustrated) through oxygen portion 256 and exiting through cannula 202 to supply oxygen 205 to patient 121 through nasal passage 165. In various embodiments, the at least a portion of adapter 100 is coupled to carbon dioxide portion 258. Carbon dioxide 210 is exhaled by patient 121 and flows from the at least a portion of adapter 100 through carbon dioxide portion 258, which may be in communication with gas analyzer 130.

With reference to FIG. 23, combination device 1280 comprises oxygen portion 256, carbon dioxide portion 258, more than one cannula 202, and more than one of the at least a portion of adapter 100. In some embodiments, the at least a portion of adapter 100 includes perforated tube 110 and flexible portion 109. In some embodiments, perforated tube 110 extends into nasal passage 165 and is configured to be positioned above the top of the cannula 202. In some embodiments of combination device 1280, each perforated tube 110 is coupled to carbon dioxide portion 258. In some embodiments of combination device 1280, each cannula 202 is coupled to oxygen portion 256. Oxygen 205 flows from source (not illustrated) through oxygen portion 256 and exiting through cannula 202 to supply oxygen 205 to patient through nasal passage 165. Carbon dioxide 210 is released by patient 121 and flows from the at least a portion of adapter 100 through carbon dioxide portion 258, which may be in communication with gas analyzer 130.

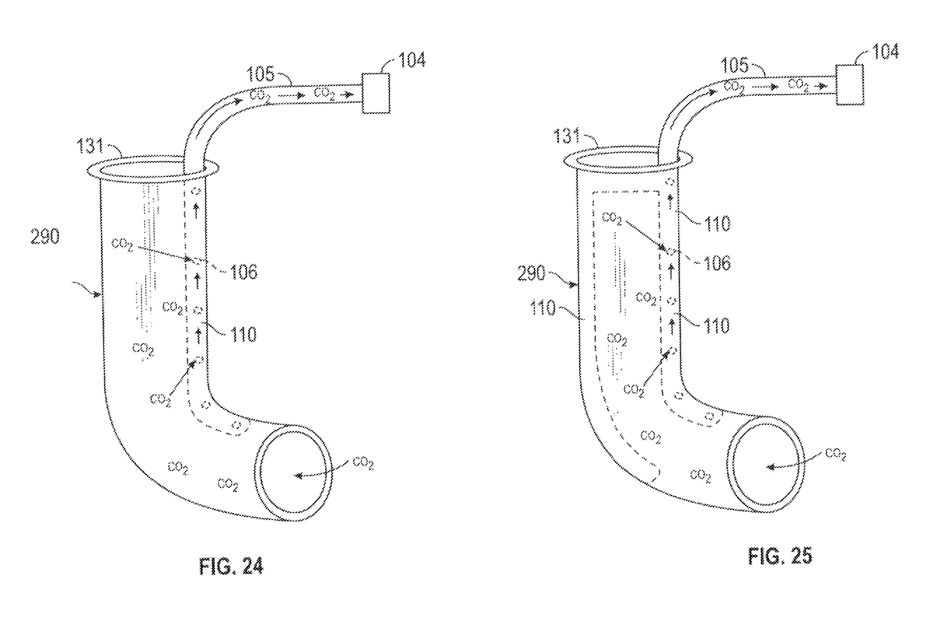

In FIGS. 24 and 25, perforated tube 110 is integrated into artificial oral airway 290, according to various embodiments. In some embodiments, perforated tube 110 is coupled to tubing 105 and connector 104. As illustrated in FIG. 25, a plurality of perforated tube 110 is integrated into artificial oral airway 290. In some embodiments, the plurality of perforated tube 110 is interconnected to each other and is in communication with tubing 105. In some embodiments, tubing 105 is configured to go over lip 131. In some embodiments, tubing 105 is configured to exit artificial oral airway 290 below lip 131. In one embodiment, tubing 105 includes fitting 107. In some embodiments, perforated tube 110 is coupled to flexible portion 109 then to tubing 105 and connector 104. In various embodiments, adapter 100 is integrated into artificial oral airway 290. In one embodiment, at least a portion of adapter 100 is integtated into artificial oral anway 290 for oropharyngeal sampinig and or monitoring. Artificial oral airway 290 comprising perforated tube 110 can be coupled to mask 125, as described herein.

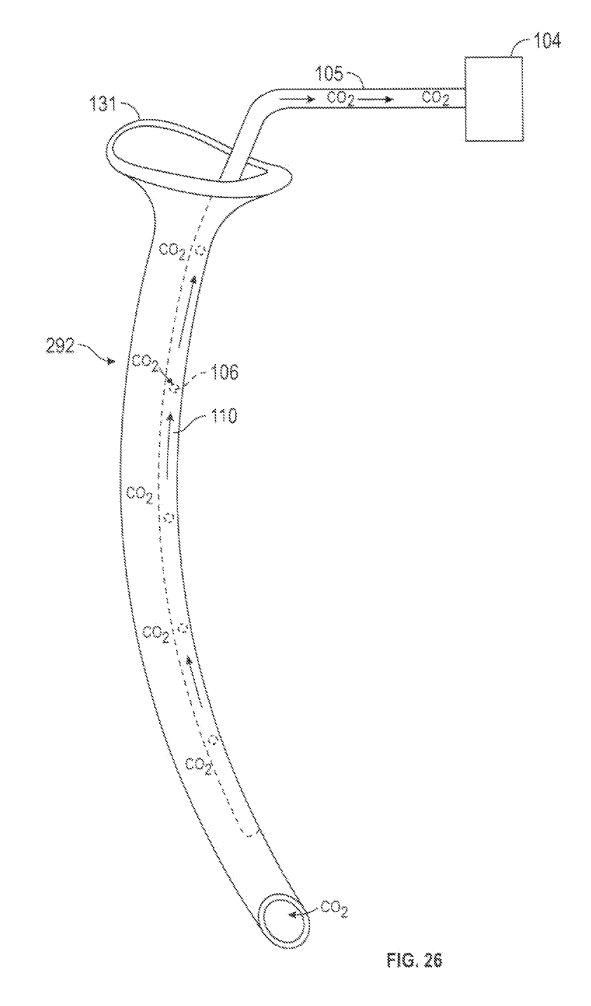

Now in FIG. 26, perforated tube 110 is integrated into artificial nasal airway 292 according to various embodiments. In some embodiments, perforated tube 110 is coupled to tubing 105 and connector 104. In some embodiments, tubing 105 is configured to go over lip 131. In some embodiments, tubing 105 is configured to exit artificial nasal airway 292 below lip 131. In some embodiments, perforated tube 110 is coupled to flexible portion 109 then to tubing 105 and connector 104. In various embodiments, adapter 100 is integrated into artificial nasal airway 292. In one embodiment, at least a portion of adapter 100 is integrated into artificial nasal airway 292 for nasopharyngeal sampling and/or monitoring. In some embodiments, a plurality of perforated tube 110 is integrated into artificial nasal airway 292. In such embodiments, the plurality of perforated tube 110 is interconnected to each other and is in communication with tubing 105. Artificial nasal airway 292 comprising perforated tube 110 can be coupled to mask 125, as described herein. Artificial nasal airway 292 comprising perforated tube 110 can be affixed to nasal cannula 202 with fastener 175, as described herein.

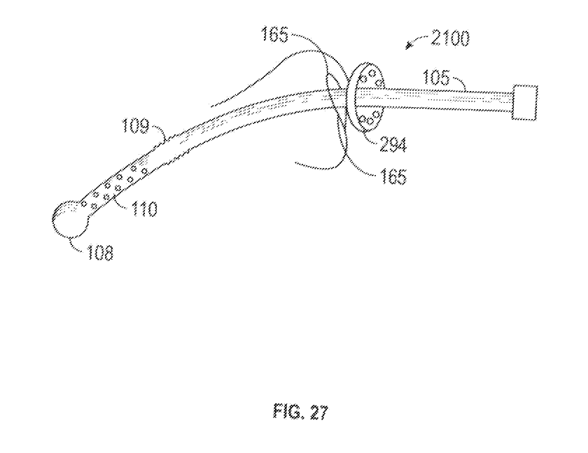

Finally with reference to FIG. 27, adapter 2100 comprises connector 104, tubing 105, bumper 294, perforated tube 110, and tip 108 according to various embodiments. In some embodiments, adapter 2100 comprises flexible portion 109 between tubing 105 and perforated tribe 110. In various embodiments, tip 108 an be weighted. In various embodiments, tip 108 can be rounded to to increase ease of inserting into nasal passage 165. In some embodiments, tip 108 is coated with a film to reduce friction. In some embodiments, tip 108 comprises a material to reduce friction. In one embodiment, tip 108 can comprise at least one hole 106 in a porton of tip 108 which is protected from nasal material entering holes as adapter 2100 is being pushed into nasal passage 165. For example, tip 108 may comprise a plurality of holes 106 in a surface closest to the perforated tube 110. In various embodiments, bumper 294 is configured to allow a predetermined portion of adapter 2100 to enter nasal passage 165. In some embodiments, bumper 294 may be movable to various locations. For example, bumper 294 may have three preset locations along adapter 2100, such as one location for juveniles, one location for smaller adults, and one location for larger adults. In some embodiments, bumper 294 is movable and lockable along adapter 2100. In some embodiments, bumper 294 comprises a plurality of perforations to allow uptake of air by patient 121 through perforations and up the nasal passage 165. In various embodiments, bumper 294 prevents tip 108 from migrating too far into nasal passage 165.

Some embodiments herein relate to methods and devices for analyzing a gas or fluid from a patient where a device as described herein is coupled to or attached to an existing oxygen delivery system or respiratory system, for example. Thus, some embodiments relate to conversion kits, devices and methods for converting technology to have the ability to better detect and analyze gases from a patient. In some embodiments, the devices or adapters can be attached, then removed. The devices can be secured in different positions to better fit the anatomy and situation of a given patient in that the lengths and positions are adjustable in many of the embodiments disclosed herein. The devices disclosed herein, in many aspects are bendable, flexible, adjustable, positionable and removeable. Thus, the devices can be provided as kits for adapting or converting existing apparatus to have added functionality or improved functionality.

As used herein, the terms “comprise”, “comprises”, “comprising”, “having”, “including”, “includes” or any variation thereof, are intended to reference a non-exclusive inclusion, such that a process, method, article, system, composition or apparatus that comprises a list of elements does not include only those elements recited, but may also include other elements not expressly listed or inherent to such process, method, article, system, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameteis or other operating requirements without departing from the general principles of the same.

In the foregoing specification, the invention has been described with reference to specific embodiments. Various modifications and changes may be made, however, without departing from the scope of the various embodiments of the present invention, as set forth in the claims. The specification and Figures are illustrative, rather than restrictive, and modifications are intended to be included within the scope of any of the various embodiments of the present invention described herein. Accordingly, the scope of the invention should be determined by the claims and their legal equivalents rather than by merely the examples described.

For example, the steps recited in any method or process claims may be executed in any order and are not limited to the specific order presented in the claims. Additionally, the components and/or elements recited in any apparatus or system claims may be assembled or otherwise operationally configured in a variety of permutations and are accordingly not limited to the specific configuration recited in the claims.

Benefits, other advantages and solutions to problems have been described above with regard to particular embodiments; however, any benefit, advantage, solution to problem or any element that may cause any particular benefit, advantage or solution to occur or to become more pronounced are not to be construed as critical, required or essential features or components of any or all the claims.

1. A system for sampling exhaled breath from a patient, the system comprising:
an at least partially flexible tube comprising an exterior surface and an interior surface, wherein a diameter of the exterior surface is less than a diameter of a hole in an oxygen supply mask configured to supply oxygen to a patient;
a connector coupled to one end of the flexible tube and configured to connect to a receiving connector on at least one of another piece of tube or a gas analyzer;
a fitting disposed around a portion of the exterior surface of the tube for attaching the flexible tube to the mask via the hole;
a sampling portion comprising a plurality of holes in a portion of the tube, wherein at least one of the plurality of holes is configured to be in communication with an interior portion of the tube; and
a shaped tip on a distal end of the tube.

2. The system according to claim 1, wherein the tube comprises a flexible portion between the connector and the sampling portion, wherein the flexible portion is configured to hold a shape.

3. (canceled)

4. The system according to claim 1, wherein the sampling portion comprises an exterior diameter essentially equal to the exterior diameter of the tube and an interior diameter essentially equal to or greater than an interior diameter of the tube.

5. The system according to claim 1, further comprising a dryer in a portion of the interior surface of the tube configured to remove moisture from the exhaled breath.

6. The system according to claim 1, wherein the shaped tip comprises an essentially smooth exterior surface and a gradient exterior shape from a high center point to a plurality of lower circumference points.

7. The system according to claim 1, wherein the shaped tip comprises a weight configured to lead the tip through a nasal passage for placement of the sampling portion into the nasal passage.

8. The system according to claim 1, wherein the shaped tip comprises at least one hole configured to be in communication with the interior portion of the tube.

9. The system according to claim 1, wherein the sampling portion is configured for placement into a portion of a nasal passage.

10. The system according to claim 1, wherein the tube is configured to communicate a portion of the exhaled air to the gas analyzer and to monitor carbon dioxide concentration.

11. The system according to claim 1, wherein the connector and the fitting are integrated.

12. The system according to claim 1, further comprising a y-shaped tube connecting the sampling portion to the tube and connecting a second sampling portion to the tube.

13. The system according to claim 12, further comprising a flexible portion integrated between at least one of the y-shaped tube and the sampling portion and between the y-shaped tube and the second sampling portion, wherein the flexible portion is configured with shape memory to hold a shape formed in the flexible portion.

14. The system according to claim 12, wherein the second sampling portion is configured in a spoon-like shape comprising a plurality of holes in communication with the y-shaped tube and is configured with the plurality of holes along an inner edge of the spoon-like shape.

15. The system according to claim 14, wherein the sampling portion s configured for placement inside a nasal passage, and the second sampling portion is configured for placement over a mouth.

16. An adapter configured to receive exhaled air from a patient, the adapter comprising:
a tube comprising an exterior surface, an interior surface and a lumen formed by the interior surface to communicate the exhaled air to a gas analyzer;
a connector coupled to one end of the tube for connecting the tube to a receiving connector on the gas analyzer;
a manifold coupled to a distal end of the tube and configured to communicate the exhaled air to the tube;
a first sampling portion comprising a plurality of holes in fluid communication with the lumen of the tube and coupled to the manifold; and
a second sampling portion comprising a plurality of holes in fluid communication with the lumen of the tube and coupled to the manifold.

17. The system according to claim 16, wherein the e second sampling portion comprises the plurality of holes around a hollow cylinder at an end distal to the manifold and having an exterior diameter essentially equal to the exterior diameter of the tube and an interior diameter essentially equal to or greater than an interior diameter of the tube, and comprising a shaped tip capping the end distal from the manifold and having an essentially smooth exterior surface, and comprises a gradient exterior shape from a high center point to a plurality of lower circumference points.

18. The system according to claim 17, wherein the first sampling portion is configured for placement into a nasal passage.

19. The system according to claim 16, wherein the second sampling portion is configured in a spoon-like shape comprising the plurality of holes along an inner edge of the spoon-like shape.

20. The system according to claim 19, wherein the first sampling portion is configured for placement inside a nasal passage, and the second sampling portion is configured for placement over a mouth.

21. The system according to claim 16, further comprising a flexible portion integrated between at least one of the manifold and the first sampling portion and between the manifold and the second sampling portion, wherein the flexible portion is configured with shape memory to hold a shape formed in the flexible portion.

22. The system according to claim 21, wherein the flexible portion comprises an exterior diameter essentially equal to the exterior diameter of the tithe and an interior diameter essentially equal to an interior diameter of the tube.

23. The system according to claim 16, wherein at least one of the first sampling portion and the second sampling portion comprises an exterior diameter essentially equal to the exterior diameter of the tube and an interior diameter essentially equal to or greater than an interior diameter of the tube.

24. The system according to claim 16, further comprising a fastener configured to affix a portion of the adapter to oxygen supply nasal cannula.

25. The system according to claim 24, further comprising the oxygen supply nasal cannula.

26. A method of measuring carbon dioxide in exhaled air from a patient, the method comprising:
coupling a tube adapter between a gas analyzer and a mask on a patient;
advancing a sampling portion of the adapter into a nasal passage of the patient;
measuring carbon dioxide levels in exhaled air from the nasal passage over time; and
converting the measured carbon dioxide levels to waveform data representing the measured levels of carbon dioxide concentration in the exhaled air from a patient over time.

27. The method according to claim 26, further comprising:
positioning a second sampling portion of the adapter over a mouth area of the patient; and
measuring carbon dioxide levels in exhaled air from the mouth.

28. The method according to claim 26, further comprising bending a shape memory portion of the adapter into a shape before the advancing step.

29. The method according to claim 26, further comprising coupling a fitting in the adapter into a hole in the mask.

30. The method according to claim 26, further comprising removing moisture in the exhaled air with a dryer in the adapter.

31. The method according to claim 26, further comprising adjusting a position of the sampling portion of the adapter in the nasal passage.

32. The method according to claim 26, wherein measuring carbon dioxide levels comprises measuring at least one carbon dioxide concentration in the exhaled air from the patient.

33. The system according to claim 1, wherein the tube includes a formable portion configured to be formed into a shape and to retain approximately the shape during use.

34. The system according to claim 33, wherein the formable portion comprises a shape memory material.

35. The system according to claim 33, wherein the formable portion comprises the shaped tip.

36. A kit for measuring exhaled breath from a patient, the system comprising:
an at least partially flexible tube comprising an exterior surface, an interior surface and an external diameter measured from one side of the exterior surface to an opposite side of the exterior surface;
a connector coupled to one end of the flexible tube and configured to connect to a receiving connector on at least one of another piece of tube or a gas analyzer;
a fitting disposed around a portion of the exterior surface of the tube for attaching the flexible tube to the mask via the hole;
a sampling portion comprising a plurality of holes in the tube from the exterior surface to the inte or surface;
a shaped tip on a distal end of the tube; and
a mask configured to cover at least one of the patient's mouth or the patient's nose, wherein the mask comprises a hole having a diameter greater than the external diameter of at least a portion of the tube.
